(19) United States
(12) Patent Application Publication
(10) Pub. No.: US 2025/0129050 A1
(43) Pub. Date: Apr. 24, 2025

(54) SYNTHESIS OF A KIF18A INHIBITOR

(71) Applicant: Amgen Inc., Thousand Oaks, CA (US)

(72) Inventors: Sebastien CAILLE, Thousand Oaks, CA (US); Daniel Gerard GREENE, Thousand Oaks, CA (US); Michael Thomas CORBETT, Thousand Oaks, CA (US); Carolyn WEI, Thousand Oaks, CA (US)

(73) Assignee: Amgen Inc., Thousand Oaks, CA (US)

(21) Appl. No.: 18/832,887

(22) PCT Filed: Jan. 26, 2023
(86) PCT No.: PCT/US2023/011636

§ 371 Date: Jul. 24, 2024

Related U.S. Application Data
(60) Provisional application No. 63/303,470, filed on Jan. 26, 2022.

(51) Int. Cl. C07D 401/14; C07D 221/20; C07F 3/00

(52) U.S. Cl. CPC C07D 401/14; C07D 221/20; C07F 3/00; C07B 2200/13

(57) ABSTRACT

The present invention relates to improved preparation of a KIF18A inhibitor having the chemical structure Compound (1), or a salt thereof Compound (la); wherein HA is as defined herein; and key intermediates thereof, i.e., Compound (2a), Compound (3a), Compound (5) or a salt thereof, and Compound (6a) or a hydrate thereof, of the formulae: Compound (2a); Compound (3a); Compound (5) or a salt thereof; and Compound (6a) or a hydrate thereof, preferably Compound (6a-1). The present invention further relates to solid form of Compound (6a), preferably the crystalline hydrate form of Compound (6a-1).

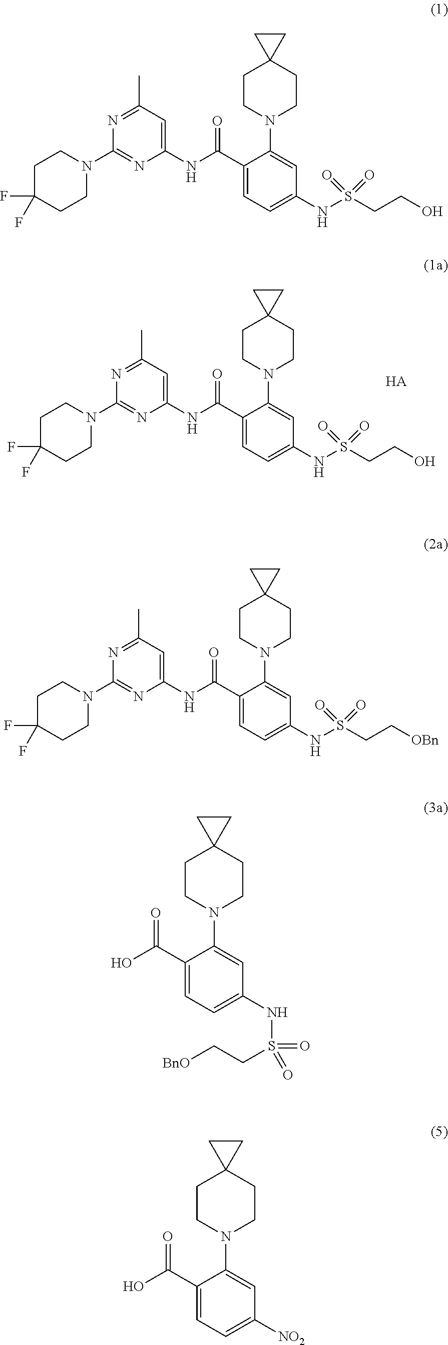 [SMILES: Cc1cc(NC(=O)c2ccc(NS(=O)(=O)CCO)cc2N2CCC3(CC2)CC3)nc(N2CCC(F)(F)CC2)n1 ; Cc1cc(NC(=O)c2ccc(NS(=O)(=O)CCO)cc2N2CCC3(CC2)CC3)nc(N2CCC(F)(F)CC2)n1 ; "HA" ; Cc1cc(NC(=O)c2ccc(NS(=O)(=O)CCOCc3ccccc3)cc2N2CCC3(CC2)CC3)nc(N2CCC(F)(F)CC2)n1 ; O=C(O)c1ccc(NS(=O)(=O)CCOCc2ccccc2)cc1N1CCC2(CC1)CC2 ; O=C(O)c1ccc([N+](=O)[O-])cc1N1CCC2(CC1)CC2]

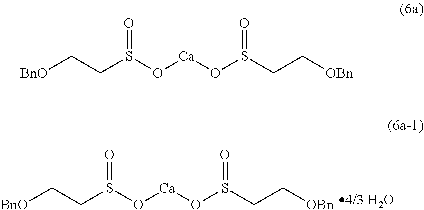

### FIELD OF THE INVENTION

The present invention relates to an improved, efficient, scalable process to prepare a KIF18A inhibitor compound, namely N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide (Compound 1), or a pharmaceutically acceptable salt thereof, which is performed in batch process, continuous process, or mixture thereof. The present invention also relates to preparation of key intermediate compounds useful for preparing the Compound 1 or a pharmaceutically acceptable salt thereof. The present invention further relates to solid form of Compound 6a, preferably the crystalline hydrate form of Compound 6a (Compound 6a-I).

### BACKGROUND OF THE INVENTION

The free base compound 2-(6-azaspiro[2.5]octan-6-yl)-N-[2-(4,4-difluoropiperidin-1-yl)-6-methylpyrimidin-4-yl]-4-[(2-hydroxyethanesulfonyl)amino]benzamide (Compound 1), or a pharmaceutically acceptable salt thereof, is useful as an inhibitor of a motor protein kinesin family member 18A (KIF18A):

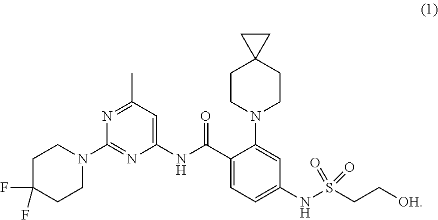 [SMILES: Cc1cc(NC(=O)c2ccc(NS(=O)(=O)CCO)cc2N2CCC3(CC2)CC3)nc(N2CCC(F)(F)CC2)n1]

Kinesins are molecular motors that play important roles in cell division and intracellular vesicles and organelle transport. Mitotic kinesin plays roles in several aspects of spindle assembly, chromosome segregation, centrosome separation, and dynamics. Human kinesins are categorized into 14 subfamilies based on sequence homology within the so-called “motor domain”; this domain's ATPase activity drives unidirectional movement along microtubules (MT). The nonmotor domain of these proteins is responsible for cargo attachment; a “cargo” can include any one of a variety of different membranous organelles, signal transduction scaffolding systems, and chromosomes. Kinesins use the energy of ATP hydrolysis to move cargo along polarized microtubules. Thus, kinesins are often called “plus-end” or “minus-end” directed motors.

KIF18A gene belongs to the Kinesin-8 subfamily and is a plus-end-directed motor. KIF18A is believed to influence dynamics at the plus end of kinetochore microtubules to control correct chromosome positioning and spindle tension. Depletion of human KIF18A leads to longer spindles, increased chromosome oscillation at metaphase, and activation of the mitotic spindle assembly checkpoint in HeLa cervical cancer cells. KIF18A appears to be a viable target for the treatment of cancer. KIF18A is overexpressed in various types of cancers, including but not limited to colon, breast, lung, pancreas, prostate, bladder, head, neck, cervix, and ovarian cancers. Further, genetic deletion or knockdown or inhibition of KIF18A affects mitotic spindle apparatus in cancer cell lines. Particularly, inhibition of KIF18A has been found to induce mitotic cell arrest, a known vulnerability that can promote cell death in mitosis via apoptosis, mitotic catastrophe, or multipolarity driven lethality or death after mitotic slippage in interphase.

Compound 1, as well as an exemplary method of making the same, is described in U.S. patent Ser. No. 11/236,069, issued Feb. 1, 2022, which is incorporated herein by reference in its entirety. The stable salt, hydrate, solvate, or co-crystal of Compound 1, along with the solid form of Compound 1 (including crystalline anhydrous Compound 1 or amorphous Compound 1), the stable salt, hydrate, solvate, or co-crystal of Compound 1, particularly for the commercial pharmaceutical production of Compound 1, are described in U.S. Provisional Patent Application No. 63/224,208, filed Jul. 21, 2021, which is incorporated herein by reference in its entirety.

U.S. patent Ser. No. 11/236,069 disclosed a method of preparing Compound 1, which relied on the introduction of key intermediate compounds in the penultimate step. It was found that the disclosed process involved a key intermediate compound that had undesirable physical properties (hygroscopicity), and had an unpredictive impurity control strategy requiring multiple re-crystallizations of the key intermediate compounds. To overcome the commercial risks identified with the disclosed method, the present inventors developed the present novel improved method to prepare Compound 1, or a pharmaceutically acceptable salt thereof, preferably HCl salt thereof, that would improve the impurity control strategy, improve convergency, scalability, and leverage key intermediate compounds with improved stability. Key intermediate compounds of the present invention that may be used in the synthesis of Compound 1 or a pharmaceutically acceptable salt thereof are Compound 2a, Compound 3a, Compound 5 or a salt thereof, and Compound 6a, of the formulae:

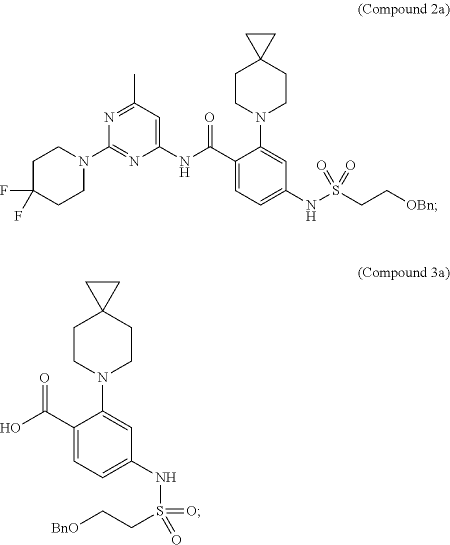 [SMILES: Cc1cc(NC(=O)c2ccc(NS(=O)(=O)CCOCc3ccccc3)cc2N2CCC3(CC2)CC3)nc(N2CCC(F)(F)CC2)n1 ; O=C(O)c1ccc(NS(=O)(=O)CCOCc2ccccc2)cc1N1CCC2(CC1)CC2]

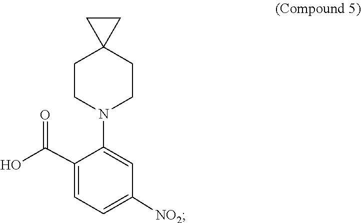 [SMILES: O=C(O)c1ccc([N+](=O)[O-])cc1N1CCC2(CC1)CC2]

or a salt thereof; and

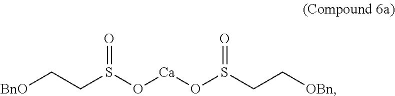

preferably the hydrate of Compound 6a:

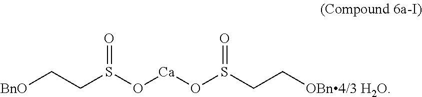

The novel key intermediate compounds are named:
(1) 4-((2-(benzyloxy)ethyl)sulfonamido)-N-(2-(4,4-difluoropiperidin-1-yl)-6-methylpyrimidin-4-yl)-2-(6-azaspiro[2.5]octan-6-yl)benzamide (Compound 2a);
(2) 4-((2-(benzyloxy)ethyl)sulfonamido)-2-(6-azaspiro[2.5]octan-6-yl)benzoic acid (Compound 3a);
(3) 4-nitro-2-(6-azaspiro[2.5]octan-6-yl)benzoic acid (Compound 5);
(4) calcium 2-(benzyloxy)ethane-1-sulfinate (Compound 6a); and
(5) calcium 2-(benzyloxy)ethane-1-sulfinate 4/3 hydrate (Compound 6a-I).

The present invention further relates to solid form of Compound 6a, preferably the crystalline hydrate form of Compound 6a (Compound 6a-I).

### SUMMARY OF THE INVENTION

In aspect 1 of the present invention, the invention provides a new method for preparing a KIF18A inhibitor having the following chemical structure:

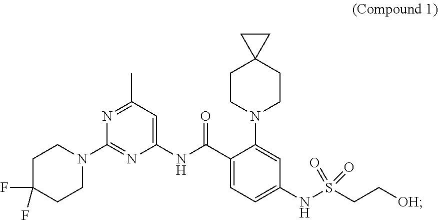 [SMILES: Cc1cc(NC(=O)c2ccc(NS(=O)(=O)CCO)cc2N2CCC3(CC2)CC3)nc(N2CCC(F)(F)CC2)n1]

comprising reacting a Compound 2 having the structure:

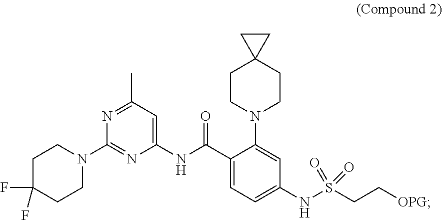

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; with a suitable deprotection agent in a suitable solvent to form said Compound 1.

In aspect 2 of the invention, the invention provides the method of aspect 1 further comprising preparing said Compound 2, comprising: reacting a Compound 3, having the formula:

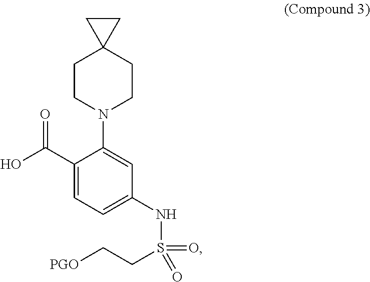

or a salt thereof; wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; with a Compound 4, having the formula

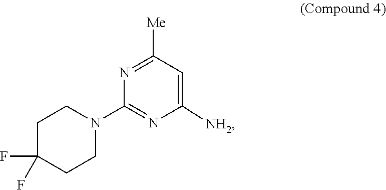 [SMILES: Cc1cc(N)nc(N2CCC(F)(F)CC2)n1]

or a salt thereof; in the presence of an amide coupling reagent and a base in an organic solvent, to form said Compound 2.

In aspect 3 of the invention, the invention provides the method of aspect 2, further comprising preparing said Compound 3, or a salt thereof, comprising: reacting a Compound 5, having the formula:

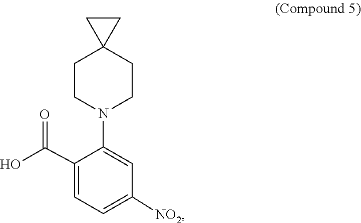 [SMILES: O=C(O)c1ccc([N+](=O)[O-])cc1N1CCC2(CC1)CC2]

or a salt thereof; with a Compound 6, having the formula

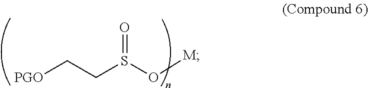

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; M is metal, including an alkali, alkaline earth metal, or transition metal, preferably sodium, calcium, or zinc metal, more preferably calcium; and n is an integer selected from 1 or 2, preferably 2; or a hydrate thereof; and a base optionally in the presence of a catalyst in an organic solvent at elevated temperature to form said Compound 3, or a salt thereof.

In aspect 4 of the invention, the invention provides the method of aspect 3, further comprising preparing said Compound 5, or a salt thereof, comprising: reacting a Compound 7, having the formula:

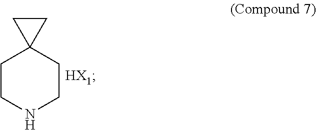

wherein X₁ is a halide, preferably chloride or bromide, more preferably chloride; with a Compound 8, having the formula

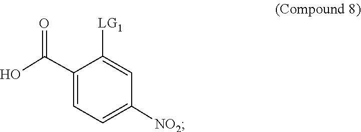

wherein LG₁ is a leaving group, preferably halo, more preferably chloro or fluoro, most preferably fluoro; in the presence of a base, in an organic solvent at elevated temperature to form said Compound 5, or a salt thereof. In aspect 5 of the invention, the invention provides the method of aspect 2, further comprising preparing said Compound 4, comprising: reacting a Compound 9, having the formula:

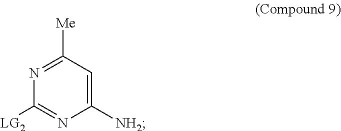

wherein LG₂ is a leaving group, preferably halo, more preferably chloro or fluoro, most preferably chloro; with a Compound 10, having the formula

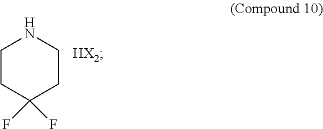

wherein X₂ is a halide, preferably chloride or bromide, more preferably chloride; in the presence of a base at elevated temperature in an organic solvent to form said Compound 4.

In aspect 6 of the invention, the invention provides a compound, which is Compound 2; wherein PG is benzyl having the structure

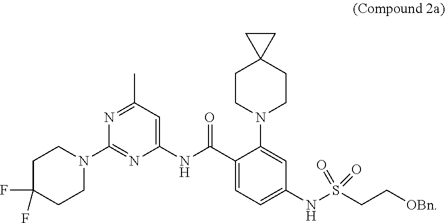 [SMILES: Cc1cc(NC(=O)c2ccc(NS(=O)(=O)CCOCc3ccccc3)cc2N2CCC3(CC2)CC3)nc(N2CCC(F)(F)CC2)n1]

In aspect 7 of the invention, the invention provides a compound, which is Compound 3; wherein PG is benzyl, having the structure:

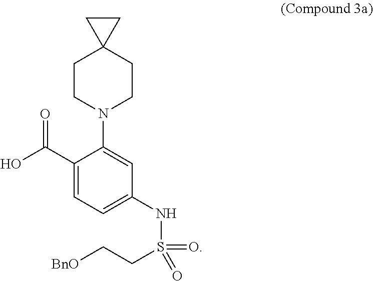 [SMILES: O=C(O)c1ccc(NS(=O)(=O)CCOCc2ccccc2)cc1N1CCC2(CC1)CC2]

In aspect 8 of the invention, the invention provides a compound, which is

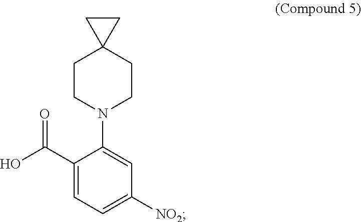 [SMILES: O=C(O)c1ccc([N+](=O)[O-])cc1N1CCC2(CC1)CC2]

or a salt thereof.

In aspect 9 of the invention, the invention provides the method of aspect 3, further comprising preparing said Compound 6 having the following chemical structure:

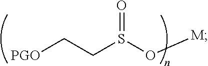

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; M is metal, including an alkali, alkaline earth metal, or transition metal, preferably sodium, calcium, or zinc metal, more preferably calcium; and n is an integer selected from 1 or 2, preferably 2; or a hydrate thereof; comprising reacting a Compound 11 having the structure

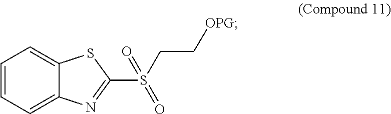

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; with a base in a polar solvent at a moderately elevated temperature to form said Compound 6; or preferably a hydrate thereof, most preferably

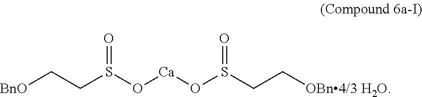

In aspect 10 of the invention, the invention provides the method of aspect 9, further comprising preparing said Compound 11 by reacting a Compound 12 having the following chemical structure:

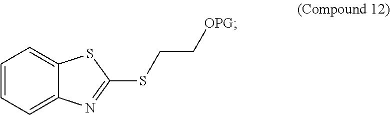

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; with an oxidizing agent; in the presence of a catalyst, in a solvent and a slightly elevated temperature to form said Compound 11.

In aspect 11 of the invention, the invention provides the method of aspect 10, further comprising preparing said Compound 12 by reacting a Compound 13 having the following chemical structure:

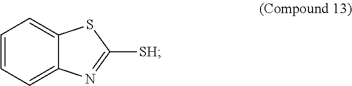

with a Compound 14 having the following chemical structure:

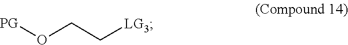

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; and LG₃ is a leaving group; preferably LG₃ is a halo, more preferably fluoro, chloro, or bromo, more preferably bromo; in the presence of a base, in a solvent and a moderately elevated temperature to form said Compound 12.

In aspect 12 of the invention, the invention provides the method of aspect 1, wherein PG is a benzyl and said suitable deprotection agent is palladium on carbon catalyst.

In aspect 13 of the invention, the invention provides the method of aspect 1, wherein said solvent is a polar solvent.

In aspect 14 of the invention, the invention provides the method of aspect 13, wherein said solvent is acetone and water mixture.

In aspect 15 of the invention, the invention provides the method of aspect 2, wherein said amide coupling reagent is selected from a chloroformamidinium salt, 2-chloro-1,3-dimethylimidazolinium chloride (DMC), 1-(chloro-1-pyrrolidinylmethylene) pyrrolidinium hexafluorophosphate (PyCIU), 2-chloro-4,6-dimethoxy-1,3,5-triazine (CDMT), or N-Ethoxycarbonyl-2-ethoxy-1,2-dihydroquinoline (EEDQ).

In aspect 16 of the invention, the invention provides the method of aspect 2, wherein said base is selected from N-methylmorpholine (NMM), N,N-Diisopropylethylamine (DIPEA), triethylamine (TEA), 2,4,6-trimethylpyridine (collidine), or 2,6-lutidine.

In aspect 17 of the invention, the invention provides the method of aspect 2, wherein said chloroformamidinium salt is chloro-N,N,N′,N′-tetramethylformamidinium hexafluorophosphate (TCFH) and said base is 2,4,6-trimethylpyridine (collidine).

In aspect 18 of the invention, the invention provides the method of aspect 2, wherein said method is conducted at a temperature from 25° C. to 50° C., preferably 40° C. to 50° C., more preferably 50° C.

In aspect 19 of the invention, the invention provides the method of aspect 2, wherein said solvent is organic solvent selected from 2-methyl THF, acetonitrile, isopropyl acetate, dichloromethane, THF, DMF, NMP, methylene chloride, or mixture thereof; preferably 2-methyl THF and acetonitrile.

In aspect 20 of the invention, the invention provides the method of aspect 3, wherein said method is performed catalyst free or in the presence of a metal catalyst selected from iron, gold, or palladium catalyst.

In aspect 21 of the invention, the invention provides the method of aspect 20, wherein said method is performed in the presence of a metal catalyst, wherein said catalyst is iron (III) chloride hexahydrate.

In aspect 22 of the invention, the invention provides the method of aspect 3, wherein said base is sodium bisulfite.

In aspect 23 of the invention, the invention provides the method of aspect 3, wherein said elevated temperature is at solvent reflux temperature or from 60° C. to 100° C.; preferably 70° C. to 90° C.; more preferably 70° C.

In aspect 24 of the invention, the invention provides the method of aspect 3, wherein said solvent is NMP.

In aspect 25 of the invention, the invention provides the method of aspect 4, wherein said base is hydroxide or amine, preferably hydroxide.

In aspect 26 of the invention, the invention provides the method of aspect 25, wherein said base is potassium hydroxide or diisopropyl ethyl amine; preferably potassium hydroxide.

In aspect 27 of the invention, the invention provides the method of aspect 4, wherein said solvent is polar aprotic solvent selected from NMP, DMAC, DMF, or DMSO; preferably NMP.

In aspect 28 of the invention, the invention provides the method of aspect 4, wherein said elevated temperature is at solvent reflux temperature or from 80° C. to 140° C.; preferably 100° C. to 140° C. C; more preferably 120° C.

In aspect 29 of the invention, the invention provides the method of aspect 4, wherein said compound 5 is crystallized by addition of an acid.

In aspect 30 of the invention, the invention provides the method of aspect 29, wherein said acid is phosphoric acid.

In aspect 31 of the invention, the invention provides the method of aspect 5, wherein said base is amine or hydroxide, preferably amine.

In aspect 32 of the invention, the invention provides the method of aspect 31, wherein said base is triethyl amine.

In aspect 33 of the invention, the invention provides the method of aspect 5, wherein said elevated temperature is at solvent reflux temperature or from 60° C. to 100° C.; preferably 60° C. to 85° C. C; more preferably 80° C.

In aspect 34 of the invention, the invention provides the method of aspect 5, wherein said solvent is acetonitrile and water mixture.

In aspect 35 of the invention, the invention provides the method of aspect 9, wherein said base is hydroxide, preferably calcium hydroxide, sodium hydroxide; more preferably calcium hydroxide.

In aspect 36 of the invention, the invention provides the method of aspect 9, wherein said solvent is a mixture of C₁₋₆alkyl alcohol and water, preferably methanol and water.

In aspect 37 of the invention, the invention provides the method of aspect 9, wherein said Compound 6 is crystallized in mixture of methanol/ethanol/water to form a hydrate Compound 6a-I having the formula

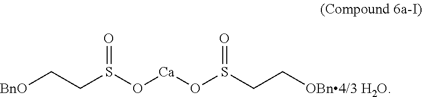

In aspect 38 of the invention, the invention provides the method of aspect 9, wherein said temperature is from 25° C. to 50° C.; from 40° C. to 50° C.; preferably 50° C.

In aspect 39 of the invention, the invention provides the method of aspect 10, wherein said oxidizing agent is peroxide or peroxycarboxylic acid; preferably hydrogen peroxide or meta peroxycarboxylic acid (mCPBA); more preferably hydrogen peroxide.

In aspect 40 of the invention, the invention provides the method of aspect 10, wherein said catalyst is sodium tungstate.

In aspect 41 of the invention, the invention provides the method of aspect 10, wherein said temperature is from 25° C. to 40° C.; preferably 30° C. to 35° C. C; more preferably 30° C.

In aspect 42 of the invention, the invention provides the method of aspect 10, wherein said solvent is acetonitrile and water mixture.

In aspect 43 of the invention, the invention provides the method of aspect 10, wherein said Compound 12 is crystallized in acetone or mixture of acetone and water.

In aspect 44 of the invention, the invention provides the method of aspect 10, wherein said Compound 12 is not isolated and said solvent is acetonitrile.

In aspect 45 of the invention, the invention provides the method of aspect 11, wherein said

base is bicarbonate, carbonate, hydroxide, or phosphate; preferably potassium carbonate, sodium carbonate, sodium hydroxide, or potassium hydroxide; more preferably potassium carbonate.

In aspect 46 of the invention, the invention provides the method of aspect 11, wherein said base is calcium carbonate.

In aspect 47 of the invention, the invention provides the method of aspect 11, wherein said solvent is alcohol, preferably methanol.

In aspect 48 of the invention, the invention provides the method of aspect 11, wherein said temperature is at solvent reflux temperature or from 70° C. to 100° C.; preferably 75° C. to 90° C. C; more preferably 82° C.

In aspect 49 of the invention, the invention provides the method of any one of aspects 1-5 or 9-48, further comprising reacting said Compound 1 with an acid HA in a solvent to form a pharmaceutically acceptable salt of Compound 1 having a formula

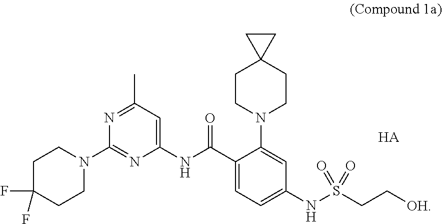 [SMILES: Cc1cc(NC(=O)c2ccc(NS(=O)(=O)CCO)cc2N2CCC3(CC2)CC3)nc(N2CCC(F)(F)CC2)n1 ; "HA"]

In aspect 50 of the invention, the invention provides the method of aspect 49, wherein said acid HX is HCl, methanesulfonic acid, or para toluenesulfonic acid.

In aspect 51 of the invention, the invention provides the method of aspect 49, wherein said acid HX is HCl in DMSO.

In aspect 52 of the invention, the invention provides the method of aspect 49, wherein said solvent is water.

In aspect 53 of the invention, the invention provides the method of any one of aspects 1-5 or 9-52, wherein said PG is benzyl.

In aspect 54 of the invention, the invention provides a compound 6 having a chemical structure:

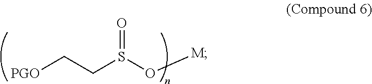

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; M is a metal, including monovalent metal, divalent metal, or trivalent metal; preferably M is monovalent metal or divalent metal; more preferably M is Na, Ca, or Zn; most preferably M is Ca; and n is an integer selected from 1, 2, or 3; preferably n is 1 or 2; more preferably n is 2; or a solid form thereof.

In aspect 55 of the invention, the invention provides the compound of aspect 54, wherein PG is benzyl, M is calcium, and n is 2 (Compound 6a), having the structure

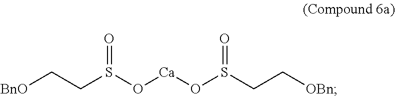

or a solid form thereof.

In aspect 56 of the invention, the invention provides the solid form of said Compound 6a according to aspect 55, which is crystalline or amorphous.

In aspect 57 of the invention, the invention provides the solid form of said Compound 6a according to aspect 55, which is a Compound 6a hydrate having the formula

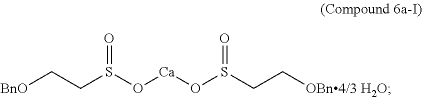

wherein said compound contains 4/3 water molecules, which is crystalline.

In aspect 58 of the invention, the invention provides the solid form of said Compound 6a according to aspect 55, which is the crystalline Form 1 of said Compound 6a hydrate (Compound 6a-I), further characterized by XRPD pattern peaks at 4.2, 8.2, and 12.2±0.2° 2θ using Cu Kα radiation.

In aspect 59 of the invention, the invention provides the solid form of said Compound 6a according to aspect 58, which is the crystalline Form 1 of said Compound 6a-I of aspect 58, further characterized by XRPD pattern peaks at 13.6, 14.2, 18.3, 19.5, 20.6, 20.9, and 22.9±0.2° 2θ using Cu Kα radiation.

In aspect 60 of the invention, the invention provides the solid form of said Compound 6a according to aspect 59, which is the crystalline Compound 6a-I of aspect 59, further characterized by XRPD pattern peaks at 16.2, 16.7, 19.2, 21.4, 23.9, 24.4, 24.7, 25.5, 27.6, 28.1, 30.3, 33.3, and 36.6±0.2° 2θ using Cu Kα radiation.

In aspect 61 of the invention, the invention provides the solid form of said Compound 6a according to aspect 60, which is the crystalline Compound 6a-I having an XRPD pattern substantially as shown in FIG. 1.

In aspect 62 of the invention, the invention provides the solid form of said Compound 6a according to aspect 58, which is the crystalline Compound 6a-I having a first endothermic transition at 124.96° C. to 130.96° C.; and a second endothermic transition at 256.11° C. to 262.11° C.; as measured by Differential Scanning calorimetry substantially as shown in FIG. 2.

In aspect 63 of the invention, the invention provides the solid form of said Compound 6a according to aspect 58, which is the crystalline Compound 6a-I wherein the first endothermic transition is at 127.96° C.±3° C.; and the second endothermic transition is at 259.11° C.±3° C.

In aspect 64 of the invention, the invention provides the solid form of said Compound 6a according to aspect 58, which is the crystalline Compound 6a-I having a Thermogravimetric Analysis (TGA) substantially as shown in FIG. 3.

In aspect 65 of the invention, the invention provides the solid form of said Compound 6a according to aspect 58, which crystalline form is stable and low in hygroscopicity. In one embodiment, the solid form stability can be identified by no change in crystalline forms and substantially identical XRPD peaks shown in the XRPD peaks of the crystalline sample pre DVS and post DVS. In another embodiment, the low in hygroscopicity feature of the crystalline sample can be shown by measuring the mass of the sample at the beginning and at the end of the DVS experiment, wherein the mass change is calculated to be between 0.10% to 0.20%; preferably 0.15%.

In aspect 66 of the invention, the invention provides the solid form of said Compound 6a according to aspect 58, which is the crystalline Compound 6a-I having a single crystal structure substantially as shown in FIG. 4.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned in the brief description of the invention and later sections herein are incorporated by reference herein in their entirety. In case of conflict, the present specification, including definitions, will control. Other features and advantages of the disclosure will be apparent from the following additional description, examples and from the claims set forth hereinbelow.

### BRIEF DESCRIPTION OF THE DRAWINGS

FIG. 1 depicts an X-ray powder diffraction (“XRPD”) pattern of (1) the crystalline Compound 6a-I pre and post humidity stress measured on Dynamic Vapor Sorption (DVS); and (2) simulated XRPD pattern from Compound 6a-I single crystal structure.

FIG. 2 depicts a Differential Scanning calorimetry (DSC) thermograph of the crystalline Compound 6a-I.

FIG. 3 depicts a Thermogravimetric analysis (TGA) of the crystalline Compound 6a-I.

FIG. 4 depicts a single crystal structure of the crystalline Compound 6a-I.

### DEFINITIONS

The following definitions should further assist in understanding the terms as used herein and the scope of the invention described herein.

The term “C_{x-y}alkyl” refers to substituted or unsubstituted saturated hydrocarbon groups, including straight-chain alkyl and branched-chain alkyl groups that contain from x to y carbons in the chain.

The term “haloalkyl” refers to alkyl groups in which at least one hydrogen atom is replace by a halo (e.g., fluoro, chloro, bromo, iodo), e.g., CH₂F, CHF₂, trifluoromethyl and 2,2,2-trifluoroethyl.

The term “comprising” is meant to be open ended, including the indicated component(s) but not excluding other elements.

The term “equivalents’ is intended to mean molar equivalents, as commonly understood by persons of ordinary skill in the art.

The term “pharmaceutically acceptable salt” refers to the relatively non-toxic, inorganic and organic acid addition salts of the compound 5 of the invention. The nature of the salt is not critical, provided that it is pharmaceutically-acceptable. These salts can be prepared in situ during the final isolation and purification of the compound(s), or by separately reacting a purified compound in its free base form with a suitable organic or inorganic acid, and isolating the salt thus formed. Suitable pharmaceutically-acceptable acid addition salts of the compound may be prepared from an inorganic acid or from an organic acid. Examples of such inorganic acids include, without limitation, hydrochloric, hydrobromic, hydroiodic, nitric, carbonic, sulfuric and phosphoric acid. Examples of organic acids include, without limitation, aliphatic, cycloaliphatic, aromatic, arylaliphatic, heterocyclic, carboxylic and sulfonic classes of organic acids, examples of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, ethanedisulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, β-hydroxybutyric, salicylic, galactaric and galacturonic acid (See, for example, Berge et al. (1977) “Pharmaceutical Salts”, J. Pharm. Sci. 66:1-19.).

As used herein, the term “hydroxyl protecting group” means a protecting group suitable for preventing undesirable reactions at a hydroxyl group. Representative hydroxyl protecting groups include, but are not limited to, silyl groups including tri (C₁₋₆alkyl) silyl groups, such as trimethylsilyl (TMS), triethylsilyl (TES), tert-butyldimethylsilyl (TBS) and the like; esters (acyl groups) including C₁₋₆alkanoyl groups, such as formyl, acetyl and the like; arylmethyl groups, such as benzyl (Bn), p-methoxybenzyl (PMB), 9-fluorenylmethyl (Fm), diphenylmethyl (benzhydryl, DPM) and the like. Numerous protecting groups, and their introduction and removal, are described in T. W. Greene and P. G. M. Wuts, Protecting Groups in Organic Synthesis, Third Edition, Wiley, New York.

As used herein, the term “metal” refers to “monovalent metal”, “divalent metal”, or “trivalent metal”.

As used herein, the term “monovalent metal” refers to a metal which is part of an ionic bond wherein said metal forms an ion having a charge of +1. Examples of monovalent metals are the metals (thus not including hydrogen) of Group 1 of the IUPAC periodic table. Preferred monovalent metals are Na, K, and Li.

As used herein, the term “divalent metal” refers to a metal which is part of an ionic bond wherein said metal forms an ion having a charge of +2. Examples of divalent metals are the metals of Group 2 of the IUPAC periodic table. Preferred divalent metals are Mg, Zn, or Ca.

As used herein, the term “trivalent metal” refers to a metal which is part of an ionic bond wherein said metal forms an ion having a charge of +3. Examples of trivalent metals are Al and Fe.

As used herein, the term “solvent” includes “aqueous solvent” or “organic solvent”.

As used herein, the term “aqueous solvent” means solvent containing water.

As used herein, the term “organic solvent” refers to an organic molecule capable of dissolving another substance (i.e., the solute). Organic solvents may be liquids at room temperature. Examples of organic solvents that may be used for the present invention include, but are not limited to: hydrocarbon solvents (e.g., n-pentane, n-hexane, n-heptane, n-octane, cyclohexane, methylcyclohexane, decahydronaphthalene, etc.) which also includes aromatic hydrocarbon solvents (e.g., benzene, toluene, o-xylene, m-xylene, and p-xylene), halogenated hydrocarbon solvents (e.g., carbon tetrachloride, 1,2-dichloroethane, dichloromethane, chloroform, etc.), ester solvents (e.g., ethyl formate, methyl acetate, ethyl acetate, isopropyl acetate, butyl acetate, isobutyl acetate, ethyl malonate, etc.), ketone solvents (e.g., acetone, methyl ethyl ketone or 2-butanone, methyl isobutyl ketone, cyclohexanone, cyclopentanone, 3-pentanone, etc.), ether solvents (e.g., diethyl ether, dipropyl ether, diphenyl ether, isopropyl ether, tert-butyl methyl ether, tetrahydrofuran, 2-methyltetrahydrofuran, 1,4-dioxane, methyl phenyl ether or anisole, etc.), amine solvents (e.g., propyl amine, diethylamine, triethylamine, aniline, pyridine), alcohol solvents (e.g., methanol, ethanol, isopropanol, 1-propanol, 2-methyl-1-propanol, 1-butanol, 2-butanol, 1-pentanol, 3-methyl-1-butanol, tert-butanol, 1-octanol, benzyl alcohol, phenol, trifluoroethanol, glycerol, ethylene glycol, propylene glycol, m-cresol, etc.), acid solvents (e.g., acetic acid, hexanoic acid, etc.), nitrobenzene, N,N-dimethylformamide, N,N,-dimethylacetamide, dimethylsulfoxide, N-methyl-2-pyrrolidone, acetonitrile, propionitrile, butyronitrile, silicone solvents (e.g., silicone oils, polysiloxanes, cyclosilicones). In some embodiments, the organic solvent may be formed by the combination of two or more organic solvents.

As used herein, the term “polar solvent” means a solvent having a dielectric constant of at least 3, said dielectric constant being the ratio of the electrical capacity of a capacitor filled with the solvent to the electrical capacity of the evacuated capacitor at 20° C. to 25° C. The values of dielectric constant of solvents are disclosed in Vogel's Textbook of Practical Organic Chemistry 5th Edition, Appendix 5. Examples of polar solvents are dichloromethane, tetrahydrofuran, ester solvents (e.g., ethyl formate, methyl acetate, ethyl acetate, ethyl malonate, etc.), ketone solvents (e.g., acetone, methyl ethyl ketone or 2-butanone, cyclohexanone, cyclopentanone, 3-pentanone, etc.), amine solvents (e.g., propyl amine, diethylamine, triethylamine, aniline, pyridine), alcohol solvents (e.g., methanol, ethanol, isopropanol, 1-propanol, 1-butanol, 1-octanol, benzyl alcohol, phenol, trifluoroethanol, glycerol, ethylene glycol, propylene glycol, m-cresol, etc.), acid solvents (e.g., acetic acid, hexanoic acid, etc.), nitrobenzene, N,N-dimethylformamide, N,N,-dimethylacetamide, dimethylsulfoxide, N-methyl-2-pyrrolidone, acetonitrile propionitrile, butyronitrile, and silicone solvents (e.g., silicone oils, polysiloxanes, cyclosilicones).

As used herein, the term “alcohol” refers to a hydrocarbon derivative in which one or more hydrogen atoms have been replaced by an —OH group, known as hydroxyl group. Suitable alcohols for the present invention include linear, cyclic or branched C₁₋₆alkyl alcohols and any mixtures thereof. It also includes commercially available alcohols. Examples of alcohols are methanol, ethanol, isopropanol, 1-propanol, 1-butanol, 1-pentanol, 3-methyl-1-butanol, tert-butanol, 1-octanol, benzyl alcohol and phenol.

As used herein, the term “benzyl” refers to a substituent group having the structure C₆H₅CH₂—.

As used herein, the term “reflux” refers to the temperature at which the reaction mixture boils, depending on the solvent being used in the reaction. For example, when water is used as a solvent, the reflux temperature is up to 100° C.

As used herein, the term “oxidizing agent” refers to a substance that can oxidize other substances, as electrons acceptor. Common oxidizing agents include oxygen (O₂); ozone (O₃); Hydrogen peroxide (H₂O₂), including other inorganic peroxides or Fenton's reagent; Organic peroxides, such as peroxycarboxylic acid having the formula RCO₃H, wherein R is an alkyl or aryl group, including peracetic acid or meta-Chloroperoxybenzoic acid (mCPBA); Fluorine (F₂), chlorine (Cl₂), or other halogens; Nitric acid (HNO₃) or nitrate compounds; Sulfuric acid (H₂SO₄); Peroxydisulfuric acid (H₂S₂O₈); Peroxymonosulfuric acid (H₂SO₅); Hypochlorite, Chlorite, chlorate, perchlorate, or other analogous halogen compounds, including household bleach (naclo); Hexavalent chromium compounds, such as chromic acids, dichromic acids, chromium trioxide, pyridinium chlorochromate (PCC), or chromate/dichromate compounds; Permanganate compounds, such as potassium permanganate (kmno4); Sodium perborate; Nitrous oxide (N₂O), Nitrogen dioxide/Dinitrogen tetroxide (NO₂/N₂O₄); Potassium nitrate (KNO₃); Sodium bismuthate (NaBiO₃); Cerium (IV) compounds, such as ceric ammonium nitrate or ceric sulfate; Lead dioxide (PbO₂); or Sodium dichromate (Na₂Cr₂O₇).

As used herein, the term “substituted” refers to moieties having substituents replacing a hydrogen on one or more non-hydrogen atoms of the molecule. Those skilled in the art will understand that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate and if allowed by valence.

As used herein, the term “continuous process” or “continuous” refers to a manufacturing process which uses one or more reagent or product stream(s) that flow continuously from one unit operation to the next, with all operations occurring simultaneously while the system is at steady state.

The term “batch process” or “batch” refers to a manufacturing process in which a given unit operation must be run to completion prior to the start of the next unit operation. Usually, the product of the prior unit operation is isolated or purified before it is used as a starting material in the next unit operation.

### GENERAL SYNTHESIS AND REPRESENTATIVE EXAMPLES OF THE INVENTION

The following abbreviations used throughout the description, including the general schemes and the examples, mean the following:

| | |
|---|---|
| DIPEA | N,N-diisopropylethylamine |
| DMF | dimethylformamide |
| DMSO | dimethyl sulfoxide |
| eq, equiv | equivalent (molar) |
| EtOAc | ethyl acetate |
| g. gm | gram |
| GC | gas chromatography |
| HPLC | high-performance liquid chromatography |
| IPAc | isopropyl acetate |
| MeCN | acetonitrile |
| MeOH | methanol |
| 2-MeTHF | 2-methyltetrahydrofuran |
| mL, ml | milliliter |
| mpk, mg/kg | milligram per kilogram |
| NaCl | sodium chloride |
| NaOH | sodium hydroxide |
| RT, rt | room temperature |
| THF | tetrahydrofuran |

### Materials and Methods

Commercially available reagents are used as is without further purification unless specified.

### PANalytical X'Pert PRO Diffractometer—Reflection Geometry

Unless specified to the contrary, XRPD patterns were collected with a PANalytical X'Pert PRO diffractometer.

Single crystal structure was determined using XtaLAB Synergy, Dualflex, HyPix diffractometer.

Differential scanning calorimetry (DSC) was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan lid was pierced by the instrument and then inserted into the DSC cell for analysis. A weighed aluminum pan configured as the sample pan was placed on the reference side of the cell.

Alternatively, Differential scanning calorimetry (DSC) analysis was also conducted on a TA Instruments Q and Discovery Series calorimeter at 10° C./min from 25° C. to 250° C. to 350° C. in an aluminum pan under dry nitrogen at 50 ml/min.

Thermal gravimetric analysis (TGA) was performed on a TA Instruments Q and Discovery Series analyzer at 10° C./min from ambient temperature between 250° C. to 350° C. in a platinum pan under dry nitrogen at 25 ml/min.

Moisture sorption data were collected using a DVS Endeavour. A sample size of about 30 mg was used. The temperature was 25° C. The relative humidity (RH) was held to 80% for 24 hours. Mass was recorded at the beginning and end of the experiment.

Solution proton NMR spectra were acquired by Spectral Data Services of Champaign (SSCI), IL at 25° C. with a Varian UNITYINOVA-400 spectrometer. Unless specified otherwise, samples were dissolved in DMSO-d6. In some cases, the solution NMR spectra were also acquired at SSCI with an Agilent DD2-400 spectrometer using deuterated DMSO or methanol.

### General Examples of the Invention

The present inventors have developed the present novel improved method of preparation of Compound 1 and a pharmaceutically acceptable salt thereof, preferably the HCl salt thereof (Compound 1a), from five key starting material/intermediate compounds: Compound 6, Compound 7, Compound 8, Compound 9, and Compound 10. The present novel route of synthesis for Compound 1 and a pharmaceutically acceptable salt of Compound 1 employs a late stage amide coupling strategy. The present method leverages the innate reactivity of 2-fluoro-4-nitrobenzoic acid (Compound 8, or a salt thereof) through sequential C—N and S—N bond formations to provide the hydroxy protected, preferably benzyl ether protected, Compound 2, which undergoes deprotection to form the free base Compound 1. Subsequent reaction of Compound 1 with acid HA in a salt formation affords a pharmaceutically acceptable salt of Compound 1.

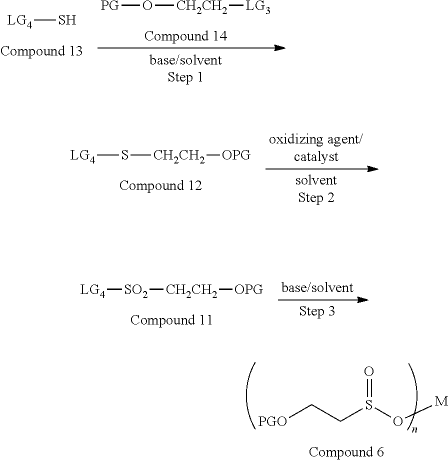

### Scheme A: Steps 1-3 Preparation of Compound 6, and Solid Form Thereof.

Scheme A depicts Steps 1-3 to prepare Compound 6 of the present invention. Compound 6 is a novel calcium sulfinate (sulfinic acid) salt compound and Scheme A describes a new, robust route to prepare calcium sulfinate salts. Literature routes to prepare sulfinate salts, for example: (a) Liang et. al., “Recent Advances in the Synthesis and Direct Application of Sulfinate Salts”, Eur. J. Org. Chem. 2020, 4664-4676; (b) Gianatassio et. al., “Simple Sulfinate Synthesis Enables C—H Trifluoromethyl-cyclopropanation”, Angew. Chem. Int. Ed. 2014, 53, 9851-9855; (c) Day et. al., “Benzothiazole Sulfinate: A Sulfinic Acid Transfer Reagent under Oxidation-Free Conditions”, Org. Lett. 2017, 19, 3819-3822; (d) Cochran et. al., “Development of a Commercial Process To Prepare AMG 232 Using a Green Ozonolysis-Pinnick Tandem Transformation”, J. Org. Chem. 2019, 84, 4763-4779; and (e) O'Hara et. al., “Preparation and purification of zinc sulfinate reagents for drug discovery”, doi:10.1038/nprot.2013.059; are known to provide facile access to sodium salts; however, most common route uses (a) reduction of sulfonyl chlorides, typically with sodium sulfite or sodium bisulfonyl chlorides as described in Scheme B below that represent handling challenges:

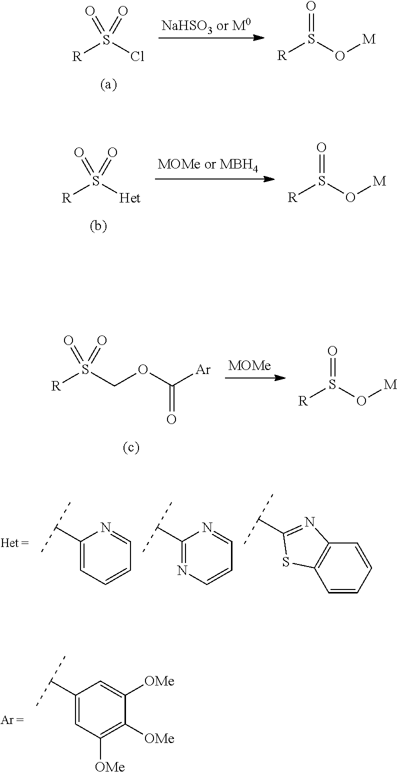

### Scheme B: Current Literature Methods to Prepare Sulfinate Salts RSO₂M; Wherein R is an Organic Functional Group; Each Het and Ar is as Defined Above; and M is Metal.

Using these literature methods, the sodium sulfinate product can be highly hygroscopic or can be found to have non-ideal physical properties. The present methods presented herein produce metal sulfinate salts, such as zinc, sodium, and calcium sulfinate salts, having improved physical properties and stability.

In Scheme A, Step 1, Compound 13, having the formula LG₄-SH, wherein LG₄ is an organic leaving group, preferably unsubstituted benzothiazolyl, is reacted with Compound 14, having the formula PG-O—CH₂CH₂-LG₃, in the presence of a base in a solvent, to form a Compound 12, having the formula LG₄-O—CH₂CH₂-OPG. Preferably, said Compound 13 has the formula Preferably, said Compound 14 has the formula

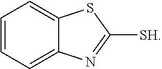

wherein PG is a hydroxyl protecting

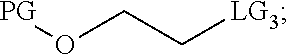

group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl. LG₃ is a leaving group; preferably LG₃ is a halo, more preferably fluoro, chloro, or bromo, more preferably bromo. Preferably, said Compound 12 has the formula

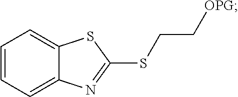

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl. Preferably, said base is bicarbonate, carbonate, hydroxide, or phosphate; preferably potassium carbonate, sodium carbonate, sodium hydroxide, or potassium hydroxide; more preferably potassium carbonate or calcium carbonate; more preferably potassium carbonate. Preferably, said solvent is alcohol, more preferably methanol. Preferably said reaction is conducted at temperature at solvent reflux temperature or from 70° C. to 100° C.; preferably 75° C. to 90° C. C; more preferably 82° C.

In an alternative, preferred, embodiment, Step 1 is performed in the presence of potassium carbonate base to generate a stable stream of said Compound 12 in acetonitrile.

In Scheme A, Step 2, Compound 12, as defined above, is reacted with an oxidizing agent; in the presence of a catalyst, in a solvent and a slightly elevated temperature to form a Compound 11. Preferably, said Compound 11 has the formula

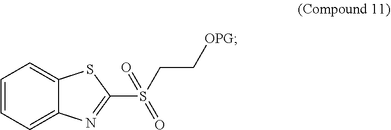

wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl. Examples of said oxidizing agent is peroxide or peroxycarboxylic acid; preferably hydrogen peroxide or meta peroxycarboxylic acid; more preferably hydrogen peroxide. Example of said catalyst is sodium tungstate. Preferably, said reaction is conducted at temperature from 25° C. to 40° C.; preferably 30° C. to 35° C. C; more preferably 30° C. Preferably, said solvent is acetonitrile and water mixture.

In one embodiment of Step 2, said Compound 12 is crystallized as crystalline solid in acetone or mixture of acetone and water.

In an alternative embodiment of Step 2, said Compound 12 was not isolated from Step 1 and said solvent is acetonitrile. Subsequent oxidation of said Compound 12 was performed with sodium tungstate as a catalyst and hydrogen peroxide as the stoichiometric oxidant.

Step 2 was a batch reaction process and faced a safety issue in using H₂O₂, which required proper safety control. To overcome the safety issue, the present inventors developed a continuous process during scale up. There were two major challenges during the initial continuous process development, one was the poor solubility of Compound 12, and the other was the slow conversion rate of the oxidation reaction of Compound 12. To overcome the solubility issue, the present inventors further screened the combined solvents volume in the continuous process and successfully used the continuous process for scale up production which improved the efficiency and safety of Step 2 production. H₂O₂ was adjusted to 30% w/w, 3.0 eq, pH range was maintained at 3-4, H₂O₂ addition was optimized to 3-4 parts, 33% NaHSO₃ solution was taken as quenching solvent.

In Scheme A, Step 3, Compound 11, as defined above, is reacted with a base in a polar solvent at a moderately elevated temperature to form said

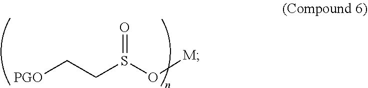

wherein PG is benzyl, M is calcium, and n is 2. Preferably, Compound 6 has the structure

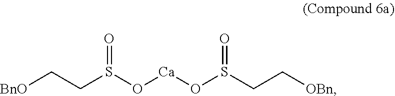

more preferably Compound 6a has the structure

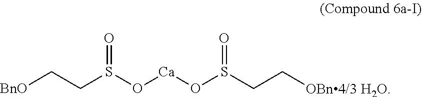

Examples of said base is hydroxide, preferably calcium hydroxide, sodium hydroxide; more preferably calcium hydroxide. Examples of said solvent is a mixture of C₁₋₆alkyl alcohol and water, preferably methanol and water; or methanol. In one embodiment, Compound 6 product is crystallized, preferably Compound 6 is crystallized in mixture of methanol/ethanol/water to form Compound 6a-I. Preferably, said reaction is conducted at temperature from 25° C. to 50° C.; from 40° C. to 50° C.; preferably 50° C. Most preferably, cleavage of the benzothiazole leaving group was accomplished employing calcium hydroxide in aqueous methanol generating the calcium sulfinate Compound 6, which was isolated via crystallization with acetone as a crystalline hydrate form. Water was found to be critical due to its ability to accelerate mass transfer of calcium hydroxide and presence in the lattice of the crystal structure of said Compound 6a. The crystallized product of Compound 6a is a crystalline hydrate compound that contains about 1 water molecule, more specifically and preferably 4/3 water molecules or about 1.33 water molecules (Compound 6a-I).

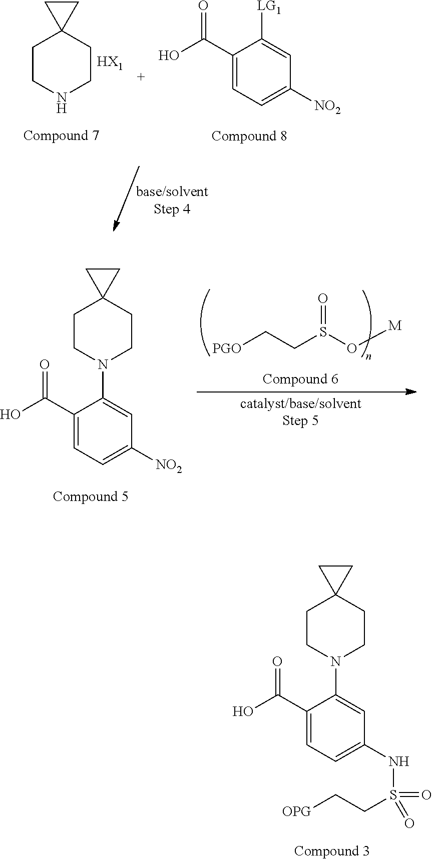

### Scheme B: Steps 4-5 Preparation of Compound 3.

Scheme B depicts Step 4 to Step 5 of the present invention. In Scheme B, Step 4, the present method leverages a base-mediated aromatic substitution reaction of Compound 7 and Compound 8 to generate a key intermediate Compound 5. The synthesis of Compound 5 is an aromatic substitution (SNAr) reaction, which is preferably performed by treatment of Compound 8; wherein LG₁ is a leaving group, preferably halo, more preferably chloro or fluoro, most preferably fluoro; and Compound 7, wherein X₁ is a halide, preferably chloride or bromide, more preferably chloride, in NMP with KOH at elevated reaction conditions. The SNAr reaction proceeds with high conversion with the desired product is directly isolated following pH adjustment with aqueous phosphoric acid and precipitation with water to afford the Compound 5 product as a crystalline solid.

In Scheme B, Step 5, subsequent nitroarene/sulfinate coupling of Compound 5 and Compound 6; or hydrate thereof (Compound 6a-I); leads to the formation of Compound 3 as a key intermediate compound. The synthesis of Compound 3 is preferably performed by an iron-catalyzed coupling of Compound 5 and Compound 6 or hydrate thereof (Compound 6a-I) in NMP with sodium bisulfite as the stoichiometric reductant. The nitroarene/sulfinate coupling reaction proceeds with high conversion and good chemo-selectivity for the desired sulfonamide product. The product is preferably isolated following aqueous work-up to purge inorganic impurities. The product is preferably solvent swapped into THF and crystallized from an MTBE anti-solvent crystallization affording Compound 3 product as a crystalline solid.

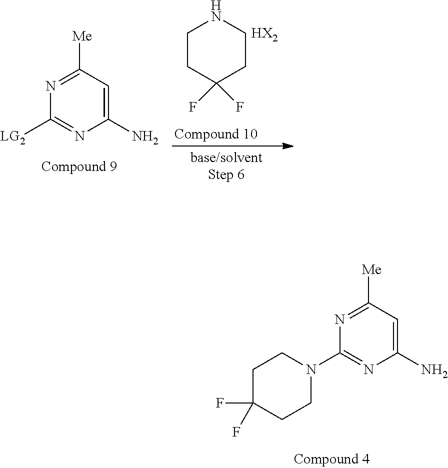

### Scheme C: Step 6 Preparation of Compound 4

Scheme C depicts Step 6 of the present invention. In this Step 6, Compound 4 is synthesized through an acid-mediated aromatic substitution reaction of Compound 9 and Compound 10. The synthesis of Compound 4 has been improved from the previously disclosed synthesis of Compound 4 in the U.S. patent Ser. No. 11/236,069. In the improved process, preferably, the Compound 4 product is generated under an acid-catalyzed SNAr reaction of Compound 9 and Compound 10, wherein X₂ is chloride, in a tert butyl alcohol: toluene solvent mixture at elevated reaction conditions. The SNAr reaction proceeds with high conversion and the desired product is isolated following salt break with aqueous NaOH and crystallization from a toluene/n-heptane solvent system to afford the product as a crystalline solid.

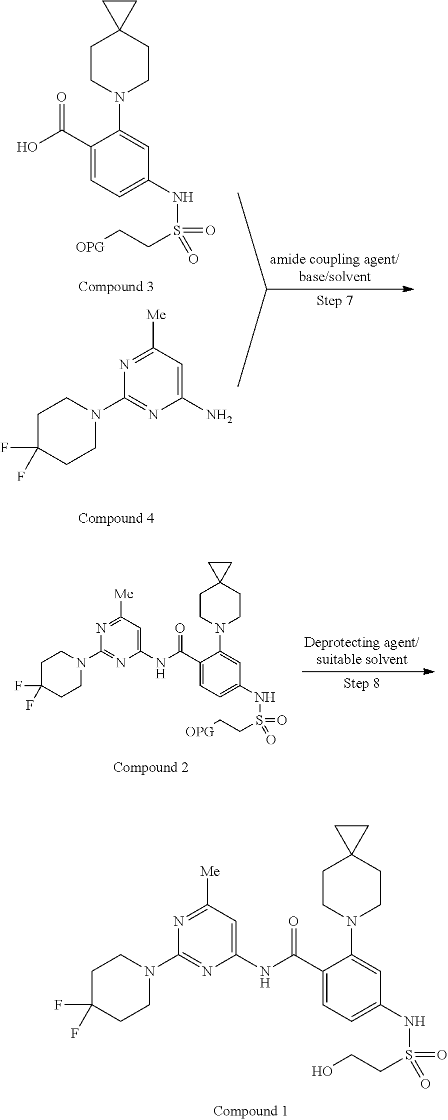

### Scheme D: Steps 7-8 Preparation of Compound 1

Scheme D depicts Steps 7-8 of the present invention to prepare Compound 1 in free base form. In Step 7, the late stage fragment coupling of Compound 3 and Compound 4 under amide-coupling conditions generates the penultimate intermediate Compound 2 as a crystalline intermediate. The synthesis of Compound 2 is preferably performed by the chloro-N,N,N′,N′-tetramethylformamidinium hexafluorophosphate (TCFH) mediated amide-coupling of Compound 3 and Compound 4 in 2-methyl THF, acetonitrile, isopropyl acetate, dichloromethane, THF, NMP, or mixture thereof; preferably 2-methyl THF and acetonitrile, in the presence of a base, preferably 2,4,6-trimethylpyridine (collidine), at elevated reaction conditions. The amide coupling proceeded with high chemoselectivity with 2,4,6-collidine as the base affording the desired amide Compound 2 product in high chemical yield following aqueous work-up and crystallization from 2-MeTHF/isopropyl amine to afford the product as a crystalline solid.

In Step 8, Pd-catalyzed hydrogenation of Compound 2 to remove the benzyl ether protecting group leads to the generation of Compound 1 as a crystalline free base compound. In the preferred embodiment, the synthesis of Compound is performed by hydrogenation of Compound 2 in the presence of Pd/C as a heterogenous catalyst in acetic acid/water mixture. The hydrogenation proceeded with high conversion and product isolation was accomplished following filtration of the catalyst and anti-solvent addition of 1-propanol to afford the Compound 1 product as a crystalline solid.

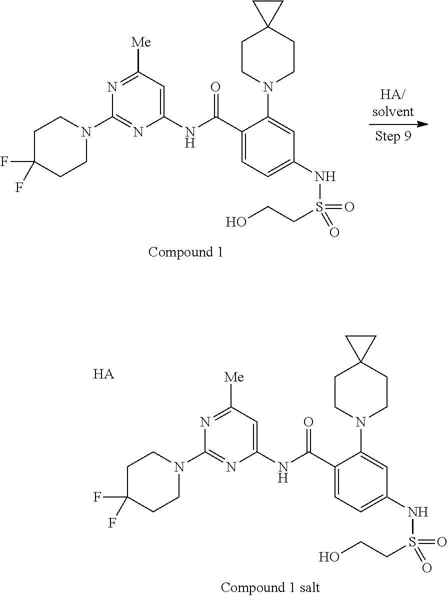 [SMILES: Cc1cc(NC(=O)c2ccc(NS(=O)(=O)CCO)cc2N2CCC3(CC2)CC3)nc(N2CCC(F)(F)CC2)n1 ; Cc1cc(NC(=O)c2ccc(NS(=O)(=O)CCO)cc2N2CCC3(CC2)CC3)nc(N2CCC(F)(F)CC2)n1 ; "HA"]

### Scheme E: Step 9 Preparation of Pharmaceutically Acceptable Salt of Compound 1

Scheme E depicts Step 9 of the present invention. In this step 9, salt formation is performed through treatment of the crystalline free base Compound 1 with HCl to generate the HCl salt of Compound 1 (Compound 1a) with optional milling which could be performed to reduce the particle size distribution (PSD). In the preferred embodiment, the synthesis of Compound 1a is performed by treatment of a solution of Compound 1 in DMSO with aqueous HCl. The product was precipitated via water anti-solvent addition to provide the product as a crystalline solid. Milling can be performed to reduce the particle size. Other preferred salts of Compound 1 include mesylate salt or tosylate salt.

### Representative Examples of the Invention

The following syntheses are representative examples of the invention and are not intended to be construed as limiting the scope of the present invention.

### Example 1: Preparation of 2-((2-(benzyloxy)ethyl)thio)benzo[d]thiazole

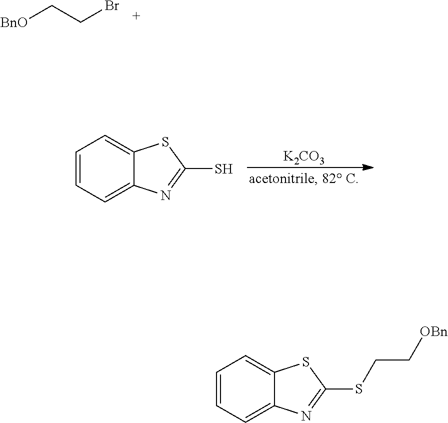

To a vial equipped with magnetic stirring was added mercaptobenzothiazole (2.8 kg, 1.00 equiv.), potassium carbonate (3.5 kg, 1.5 equiv.) and anhydrous acetonitrile (22 L). ((2-Bromoethoxy)methyl)benzene (3.9 L, 1.00 equiv.) was added to the agitated solution dropwise. The solution was agitated at 82° C. for 18 hours. The mixture was filtered on a medium porosity glass fritted funnel to remove solids and the filtrate was concentrated to afford the product 2-((2-(benzyloxy)ethyl)thio)benzo[d]thiazole as an orange colored oil, which was used in next step without further purification.

### Example 2: Preparation of 2-((2-(benzyloxy)ethyl)sulfonyl)benzo[d]thiazole

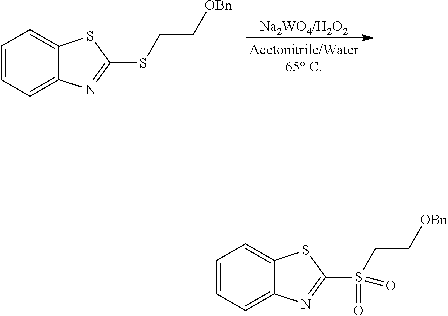 [SMILES: c1ccc(COCCSc2nc3ccccc3s2)cc1 ; "Na2WO4/H2O2" ; O=S(=O)(CCOCc1ccccc1)c1nc2ccccc2s1]

Three solutions in separate plug flow reactors were combined in a mixer and circulated in a single plug flow reactor with a residence time of 15 minutes at 50° C. The three solutions were prepared as follows: (i) 2-((2-(benzyloxy)ethyl)thio)benzo[d]thiazole (5.0 kg, 1.0 equiv.) in acetonitrile (27.5 L), (ii) Na₂WO₄ (245 g, 0.05 equiv.), water (2.5 L), phosphoric acid (85%, 125 g, 0.025 equiv.), and (iii) Aqueous H₂O₂ (1.8 kg of 35% w/w solution, 4.5 equiv.). The solution was passed through a continuously stirred-tank reactor for 10 min at 60° C. followed by another plug flow reactor for 15 min at 65° C. The mixture was cooled to 20° C. and 55 L of water were added. Aqueous NaHSO₃ (10 L of 33% w/w solution, 1.9 equiv.) was added while maintaining the temperature at 20° C. The mixture was filtered, and the cake washed twice with 15 L of water. The cake was washed with 15 L of heptane and dried at 40° C. under vacuum to yield the product 2-((2-(benzyloxy)ethyl)sulfonyl)benzo[d]thiazole.

### Example 3: Preparation of calcium 2-(benzyloxy)ethane-1-sulfinate and the crystalline hydrate salt thereof

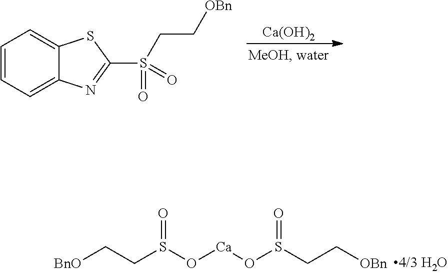

To a mixture of 2-((2-(benzyloxy)ethyl)sulfonyl)benzo[d]thiazole (4.4 kg, 1.0 equiv.) and Ca(OH)₂ (98 g, 0.1 equiv.) in methanol (26 L) at 50° C. was added Ca(OH)₂ (431 g, 0.44 equiv.) in 4 portions over the course of 8 hours. The mixture was cooled to 40° C. and filtered. The cake was washed with 18 L of methanol. The filtrate was concentrated to approximately 9 L of solution and warmed to 55° C. Water (2.2 L) and then ethanol (16.5 L) were added at 55° C. The mixture was cooled to 5° C. over 4 hours and agitated at that temperature for 10 hours. The slurry was filtered and the cake washed with 22 L of ethanol. To the cake was added 4.4 L of methanol and the mixture was warmed to 55° C. Water (1.1 L) and then ethanol (16.5 L) were added at 55° C. The mixture was cooled to 5° C. over 4 hours and agitated at that temperature for 10 hours. The slurry was filtered and the cake washed with 44 L of ethanol. The cake was dried at 40° C. under vacuum, leading to isolation of calcium 2-(benzyloxy)ethane-1-sulfinate as a crystalline hydrate, more specifically 4/3 hydrate compound, having the formula

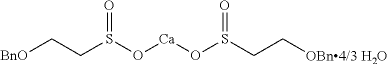

in 72% yield. 1H NMR (500 MHZ, D₂O): δ 7.43 (m, 10H), 4.58 (s, 4H), 3.84 (t, 6.2 Hz, 4H), 2.62 (t, 6.2 Hz, 4H); ¹³C NMR (125 MHZ, D₂O): δ 138.3, 129.8, 129.5, 129.4, 74.0, 65.8, 62.3. HRMS-ESI (m/z): [M]⁻ calcd for [C₉H₁₁O₃S]⁻, 199.0434; found: 199.0429.

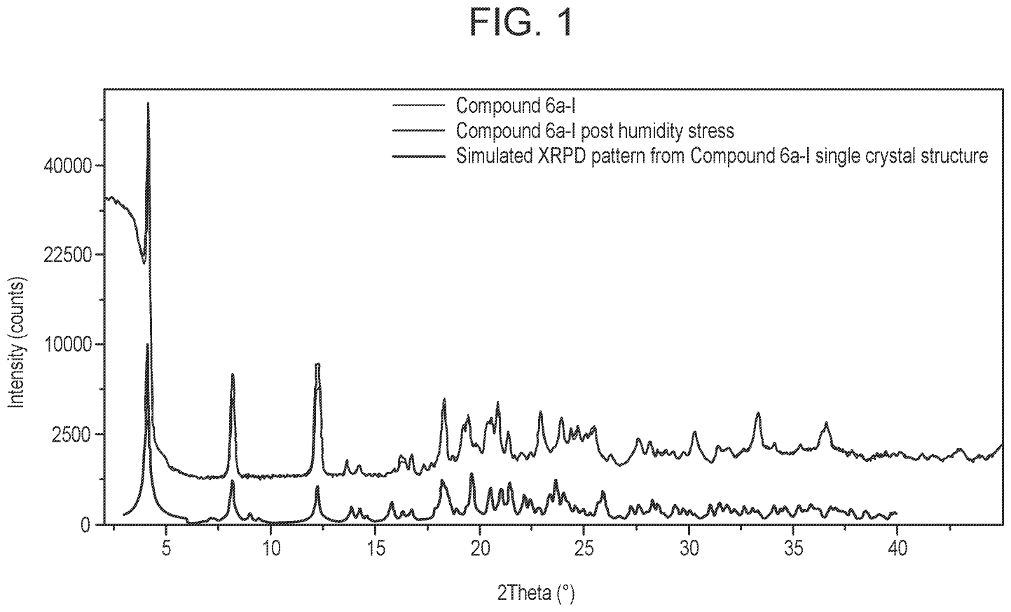

X-Ray Powder Diffraction: The XRPD pattern of the Crystalline Compound 6a-I is shown in FIG. 1. Samples were scanned at ambient temperature in continuous mode from 5-45 degrees or 2-45 degrees (2θ) with step size of 0.0334 degrees at 45 kV and 40 mA with CuKα radiation (1.54 Å). The incident beam path was equipped with a 0.02 radian soller slit, 15 mm mask, 4 degrees fixed anti-scatter slit, and a programmable divergence slit. The diffracted beam was equipped with a 0.02 rad soller slit, programmable anti-scatter slit and a 0.02 mm nickel filter. Samples were prepared on a low background sample holder and placed on a spinning stage with a rotation time of 2 seconds.

Stability and Hygroscopicity Features of Crystalline Compound 6a-I as shown by Pre and Post DVS XRPD Experiment: Crystalline Compound 6a-I was exposed to 80% relative humidity at 25° C. for 24 hours using a DVS Endeavor instrument. The mass was recorded at the beginning and end of the exposure. The present inventors found that the starting mass was 32.3989 mg, whereas the ending mass was 32.4479, which is calculated to be 0.15% mass change. The mass change indicated that Crystalline Compound 6a-I absorbed 0.15% water by weight. This shows that the Crystalline Compound 6a-I has low hygroscopicity. There was no crystalline form change found after exposure to 80% relative humidity at 25° C. for 24 hours as shown by the substantially identical XRPD peaks of crystalline samples pre DVS and post DVS (See Tables 1 and 2).

TABLE 1
XRPD pattern of the Crystalline
Compound 6a-I taken pre-DVS experiment.

| | Pos. | FWHM | Height | Rel. Int. |
|---|---|---|---|---|
| No. | [°2Th.] | [°2Th.] | [cts] | [%] |
| 1 | 4.1653 | 0.1299 | 39075.02 | 100 |
| 2 | 8.2116 | 0.1624 | 4207.16 | 10.77 |
| 3 | 12.34 | 0.2273 | 4923.88 | 12.6 |
| 4 | 13.6332 | 0.1299 | 535.07 | 1.37 |
| 5 | 14.2557 | 0.1624 | 316.12 | 0.81 |
| 6 | 16.2346 | 0.1624 | 491.17 | 1.26 |
| 7 | 16.7384 | 0.1299 | 755.66 | 1.93 |
| 8 | 17.3456 | 0.0974 | 308.55 | 0.79 |
| 9 | 17.6805 | 0.0974 | 387.59 | 0.99 |
| 10 | 18.3073 | 0.1948 | 3991.79 | 10.22 |
| 11 | 19.1854 | 0.1299 | 2184.57 | 5.59 |
| 12 | 19.4693 | 0.1299 | 2785.87 | 7.13 |
| 13 | 20.3308 | 0.1624 | 2100.84 | 5.38 |
| 14 | 20.5611 | 0.0974 | 2608.4 | 6.68 |
| 15 | 20.8654 | 0.1624 | 3726.21 | 9.54 |
| 16 | 21.3653 | 0.1624 | 1681.86 | 4.3 |
| 17 | 22.9044 | 0.1624 | 2957.51 | 7.57 |
| 18 | 23.9235 | 0.1624 | 2482.97 | 6.35 |
| 19 | 24.3679 | 0.0974 | 1537.92 | 3.94 |
| 20 | 24.6964 | 0.1624 | 1725.59 | 4.42 |
| 21 | 25.0744 | 0.0974 | 1504.37 | 3.85 |
| 22 | 25.5153 | 0.1299 | 1884.08 | 4.82 |
| 23 | 26.313 | 0.2598 | 412.31 | 1.06 |
| 24 | 27.6446 | 0.2273 | 1078.85 | 2.76 |
| 25 | 28.1375 | 0.1624 | 1009.84 | 2.58 |
| 26 | 29.7416 | 0.1948 | 424.37 | 1.09 |
| 27 | 30.3171 | 0.1624 | 1398.98 | 3.58 |
| 28 | 31.4054 | 0.1948 | 699.03 | 1.79 |
| 29 | 31.9372 | 0.2598 | 512.25 | 1.31 |
| 30 | 33.3317 | 0.2598 | 2387.17 | 6.11 |
| 31 | 34.0836 | 0.2598 | 622.63 | 1.59 |
| 32 | 35.3455 | 0.1299 | 627.14 | 1.6 |
| 33 | 36.607 | 0.2273 | 1673.59 | 4.28 |
| 34 | 40.0737 | 0.5196 | 250.95 | 0.64 |
| 35 | 41.166 | 0.1948 | 237.1 | 0.61 |
| 36 | 43.0567 | 0.4546 | 413.46 | 1.06 |
| 37 | 44.0271 | 0.396 | 158.02 | 0.4 |

TABLE 2
XRPD pattern of the Crystalline Compound
6a-I taken post-DVS experiment.

| No. | Pos. [°2Th.] | Height [cts] | Rel. Int. [%] |
|---|---|---|---|
| 1 | 4.1441 | 53251.93 | 100 |
| 2 | 8.1832 | 6260.19 | 11.76 |
| 3 | 12.1669 | 6996.7 | 13.14 |
| 4 | 12.3196 | 6790.37 | 12.75 |
| 5 | 13.6248 | 505.22 | 0.95 |
| 6 | 14.233 | 337.21 | 0.63 |
| 7 | 16.2008 | 747.17 | 1.4 |
| 8 | 16.7253 | 781.98 | 1.47 |
| 9 | 17.3306 | 380.13 | 0.71 |
| 10 | 18.2905 | 3746.49 | 7.04 |
| 11 | 19.1744 | 2165 | 4.07 |
| 12 | 19.4439 | 2754.62 | 5.17 |
| 13 | 20.3266 | 2439.46 | 4.58 |
| 14 | 20.5549 | 2667.5 | 5.01 |
| 15 | 20.8519 | 3455.68 | 6.49 |
| 16 | 21.3579 | 1820.7 | 3.42 |
| 17 | 22.0042 | 852.91 | 1.6 |
| 18 | 22.434 | 907.63 | 1.7 |
| 19 | 22.902 | 3012.66 | 5.66 |
| 20 | 23.9131 | 2758.48 | 5.18 |
| 21 | 24.3522 | 2127.17 | 3.99 |
| 22 | 24.7059 | 2278.69 | 4.28 |
| 23 | 25.4868 | 2153.79 | 4.04 |
| 24 | 26.2955 | 701.52 | 1.32 |
| 25 | 27.6141 | 1507.92 | 2.83 |
| 26 | 28.1498 | 1353.89 | 2.54 |
| 27 | 29.297 | 898.71 | 1.69 |
| 28 | 30.2733 | 1927.7 | 3.62 |
| 29 | 31.3433 | 1144.97 | 2.15 |
| 30 | 31.9218 | 1071.39 | 2.01 |
| 31 | 33.3261 | 3075.86 | 5.78 |
| 32 | 34.0915 | 1335.79 | 2.51 |
| 33 | 35.3497 | 1253.02 | 2.35 |
| 34 | 36.6064 | 2479.01 | 4.66 |
| 35 | 38.0256 | 793.66 | 1.49 |
| 36 | 38.9685 | 772.68 | 1.45 |
| 37 | 40.1748 | 893.34 | 1.68 |
| 38 | 41.1346 | 829.97 | 1.56 |
| 39 | 42.9912 | 1058.13 | 1.99 |
| 40 | 43.884 | 784.72 | 1.47 |

XPRD Peak Characterizations: The crystalline Compound 6a-I was further characterized by an X-ray powder diffraction pattern (XPRD) and was found to be a stable polymorph form, having peaks at 4.2, 8.2, and 12.2=0.2° 2θ using Cu Kα radiation. The crystalline Compound 6a-I optionally can be further characterized by an X-ray powder diffraction pattern having additional peaks at 13.6, 14.2, 18.3, 19.5, 20.6, 20.9, and 22.9±0.2° 2θ using Cu Kα radiation. The crystalline Compound 6a-I optionally can be further characterized by an X-ray powder diffraction pattern having additional peaks at 16.2, 16.7, 19.2, 21.4, 23.9, 24.4, 24.7, 25.5, 27.6, 28.1, 30.3, 33.3, and 36.6±0.2° 2θ using Cu Kα radiation. In some embodiments, crystalline Compound 6a-I has an X-ray powder diffraction pattern substantially as shown in FIG. 1, wherein by “substantially” is meant that the reported peaks can vary by ±0.2°. Those skilled in the art know that in the field of XRPD, while relative peak heights in spectra are dependent on a number of factors, such as sample preparation and instrument geometry, peak positions are relatively insensitive to experimental details.

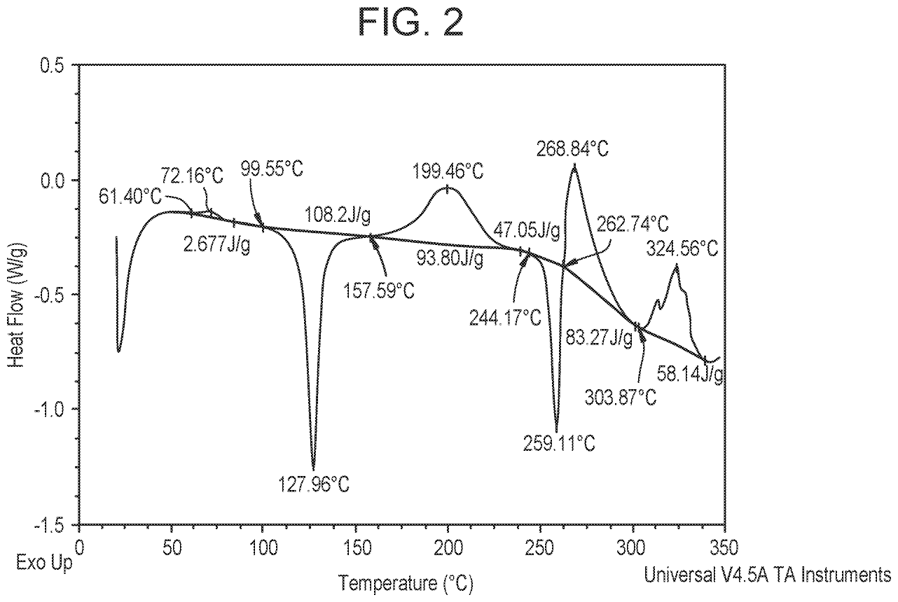

Differential scanning calorimetry (DSC) thermographs were obtained for the crystalline Compound 6a-I. The DSC curve indicates an endothermic transition at 127.96° C.±3° C. Thus, in some embodiments, the crystalline Compound 6a-I can be characterized by a DSC thermograph having a transition endotherm with an onset of 124.96° C. to 130.96° C. For example, in some embodiments the crystalline Compound 6a-I is characterized by DSC, as shown in FIG. 2.

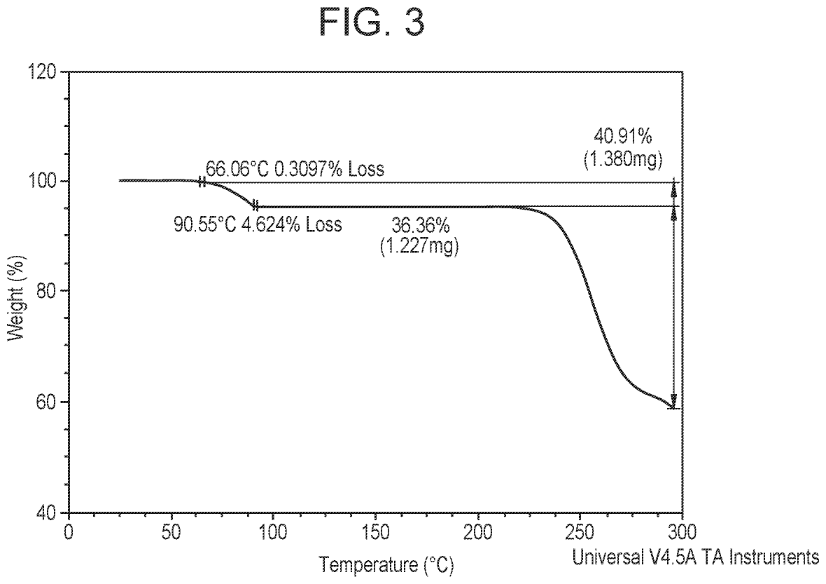

Thermogravimetric Analysis (TGA): The crystalline Compound 6a-I also can be characterized by TGA. Thus, the crystalline Compound 6a-I can be characterized by a weight loss in a range of about 4.5% to about 4.7% with an onset temperature of 124.96° C. to 130.96° C. For example, the crystalline Compound 6a-I can be characterized by a weight loss of about 4.62%, up to about 127.96° C. In some embodiments, the crystalline Compound 6a-I has a thermogravimetric analysis substantially as depicted in FIG. 3, wherein by “substantially” is meant that the reported TGA features can vary by +3° C.

Dynamic Vapor Sorption (DVS): The crystalline Compound 6a-I can be characterized by a DVS moisture sorption profile. XPRD peaks characterization pre and post DVS showed that the crystalline Compound 6a-I has a stable crystalline polymorph form and is low in hygroscopicity, in that the weight loss, which corresponded to water loss, was only 0.15%.

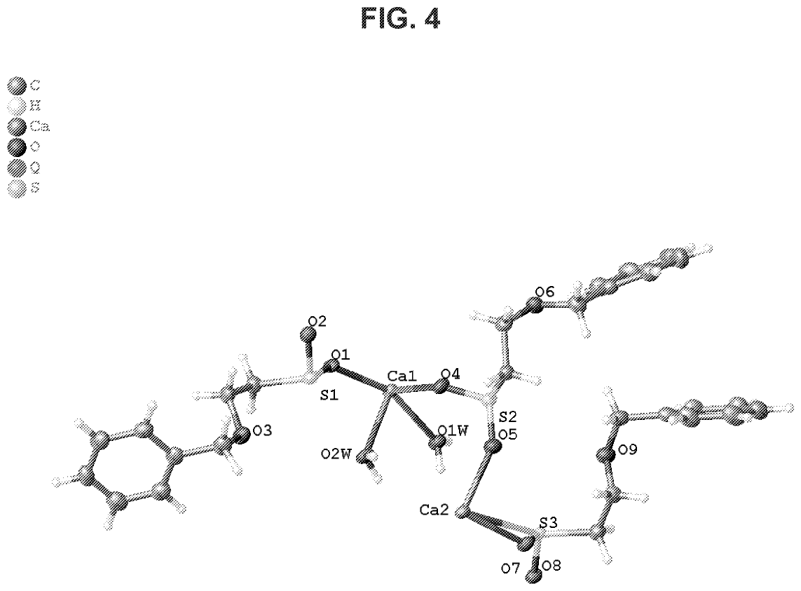

Single Crystal Experimental: Single colorless needle-shaped crystals of Compound 6a-I was used. A suitable crystal with dimensions 0.23× 0.09×0.01 mm3 was selected and mounted on a mylar loop with paratone oil on a XtaLAB Synergy, Dualflex, HyPix diffractometer. The crystal was kept at a steady T=100.00 (10) K during data collection. The structure was solved with the ShelXT 2018/2 (Sheldrick, 2018) solution program using dual methods and by using Olex2 1.5-alpha (Dolomanov et al., 2009) as the graphical interface. The model was refined with ShelXL 2018/3 (Sheldrick, 2015) using full matrix least squares minimisation on F₂. Table 3 shows the crystallographic data summary of the crystalline Compound 6a-I as shown in FIG. 4.

TABLE 3
X-ray Single Crystallographic data summary
of the Crystalline Compound 6a-I:

| Crystallization conditions | Slow diffusion from | |
|---|---|---|
| | methanol and water | |
| Crystal morphology | Needle (colorless) | |
| Crystal dimensions (mm³) | 0.233 × 0.085 × 0.015 | |
| Chemical formula | C₂₇H₃₇Ca_{1.5}O₁₁S₃ | |
| Form | Crystalline form 1 | |
| T (data collection) | 100.00 | (10) K |
| Crystal system/space group | P −1 (2) | |
| Unit cell dimensions | a = 5.7266 (2) Å | α = 103.423° (3) |
| (Å, °) | b = 12.9146(5) Å | β = 93.752° (3) |
| | c = 22.4499(8) Å | γ =97.300° (3) |
| Unit cell volume (Å³) | 1594.10 | (10) |
| Z (Z′) | 2 | (1) |
| Calculated density (mg/m3) | 1.446 | |
| R1 (all data) | 0.0728 | (0.0830) |

### Example 4: Preparation of 4-nitro-2-(6-azaspiro[2.5]octan-6-yl)benzoic acid

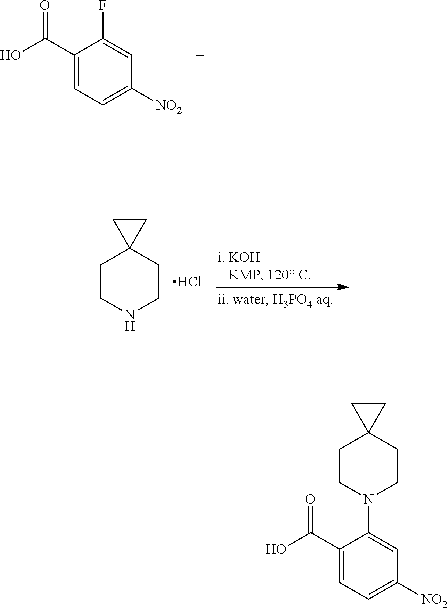 [SMILES: O=C(O)c1ccc([N+](=O)[O-])cc1F ; C1CC2(CCN1)CC2 ; Cl ; O=C(O)c1ccc([N+](=O)[O-])cc1N1CCC2(CC1)CC2]

To a 2 L vessel was added 2-fluoro-4-nitrobenzoic acid (150 g, 1.0 equiv.), 6-azaspiro[2.5]octane hydrochloride (131.6 g, 1.10 equiv.), and NMP (900 ml). The resultant mixture was stirred at 20° C. for 30 minutes. KOH (112.3 g, 2.10 equivalents) was added and the mixture was heated to 120° C. The mixture was agitated at 120° C. for 16 hours and cooled to 90° C. Aqueous H₃ PO₄ (0.6 equiv H₃PO₄ in 750 ml of water) at 90° C. over 2 hours. After addition, the suspension was agitated at 90° C. for 1 hour, cooled to 20° C. over 5 hours, and agitated at 20° C. for 5 hours. The product was isolated by filtration, washed with a 1/1 mixture of NMP/water (500 mL) followed by water (500 mL). The cake was dried under vacuum to afford 4-nitro-2-(6-azaspiro[2.5]octan-6-yl)benzoic acid as a crystalline solid in 90% yield. 1H NMR (500 MHZ, dmso-d6): δ 15.46 (bs, 1H), 8.15 (bs, 1H), 7.98 (bd, 8.6 Hz, 1H), 7.94 (bd, 8.9 Hz, 1H), 3.17 (m, 4H), 1.53 (m, 4H), 0.39 (s, 4H); ¹³C NMR (125 MHZ, dmso-d6): δ 166.8, 151.6, 149.8, 131.8, 130.5, 117.7, 115.6, 52.1, 34.4, 16.6, 11.2; HRMS-ESI (m/z): [M+H]⁺ calcd for [C₁₄H₁₆N₂O₄+H]⁺, 277.1183; found, 277.1183.

### Example 5: Preparation of 4-((2-(benzyloxy)ethyl)sulfonamido)-2-(6-azaspiro[2.5]octan-6-yl)benzoic acid

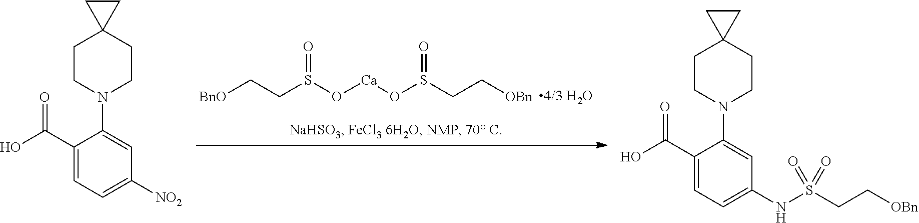

4-nitro-2-(6-azaspiro[2.5]octan-6-yl)benzoic acid (150 g, 1.0 equiv), calcium 2-(benzyloxy)ethane-1-sulfinate (143 g, 0.6 equiv), NaHSO₃ (226 g, 4.0 equiv), FeCl₃·6H₂O (14.5 g, 0.1 equiv) was added to a reaction vessel. NMP (750 mL) was added. The mixture was warmed to 70° C. over 1 hour and agitated at that temperature for 8 hours. The batch was cooled to 50° C. and diluted with THF (2.25 L). Aqueous EGTA (20%), adjusted to pH 7 with 10 M NaOH (1.05 L) and 20% w/w aqueous sodium chloride (450 mL) were added. The mixture was warmed to 50° C. prior to pH adjusted to 7-8 with 10 M NaOH. The mixture was agitated for 30 minutes and the layers were allowed to separate. The lower aqueous layer was drained. 20% w/w Aqueous sodium chloride (1.5 L) was added and the contents were warmed to 50° C. The pH was adjusted to 5 with 6M HCl. The mixture was agitated for 20 minutes, the layers allowed to separate, and the lower aqueous layer drained. 20% w/w Aqueous sodium chloride (1.5 L) was added and the contents were warmed to 50° C. The mixture was agitated for 20 minutes, the layers allowed to separate, and the lower aqueous layer drained. 20% w/w Aqueous sodium chloride (1.5 L) was added and the contents were warmed to 50° C. The mixture was agitated for 20 minutes, the layers allowed to separate, and the lower aqueous layer drained. The organic layer was distilled under vacuum from 3 L to 750 mL twice while adding fresh THF. The organic layer was diluted to 3 L with THF and polish filtered. The organic layer was concentrated to 750 mL. The mixture was heated to 50° C., ensuring full dissolution. The mixture was cooled to 40° C. over 1 hour. At 40° C., 4-((2-(benzyloxy)ethyl)sulfonamido)-2-(6-azaspiro[2.5]octan-6-yl)benzoic acid seed, which was obtained from previous batch, (2.5 wt %) was added and the slurry was agitated for 1 hour. Toluene (5 L/kg) was added at 40° C. over 5 hours. The mixture was heated to 50° C. and agitated for 1 hour. Toluene (5 L/kg) was added at 50° C. over 3 hours. The suspension was cooled to 20° C. over 12 hours and agitated 1 hour. The solid product was filtered. The cake was washed with pre-mixed 1:2 THF/toluene (900 mL) and then with toluene (900 mL). The cake was vacuum dried under a stream of nitrogen at 40° C. and the product 4-((2-(benzyloxy)ethyl)sulfonamido)-2-(6-azaspiro[2.5]octan-6-yl)benzoic acid was isolated as a crystalline solid in 79% yield. Without the crystal seed, the yield was about 1% less. Melting point: 150.5-164.7° C.; 1H NMR (500 MHZ, dmso-d6): δ 10.38 (br, 1H), 7.24-7.37 (m, 4H), 7.19-7.23 (m, 2H), 7.17 (dd, 1H), 4.42 (s, 2H), 3.79 (t, 2H), 3.57 (t, 2H), 2.94 (t, 4H), 1.55 (br, 4H), 0.41 (s, 4H); ¹³C NMR (125 MHz, dmso-d6): δ 166.7, 152.8, 138.2, 132.9, 128.6, 127.9, 119.4, 116.5, 111.8, 72.4, 64.2, 53.7, 52.0, 34.9, 16.9, 11.7; HRMS-ESI (m/z): [M+H]⁺ calcd for [C₂₃H₂₈N₂O₅S+H]⁺, 445.1792; found: 445.1782.

### Example 6: Preparation of 2-(4,4-difluoropiperidin-1-yl)-6-methylpyrimidin-4-amine

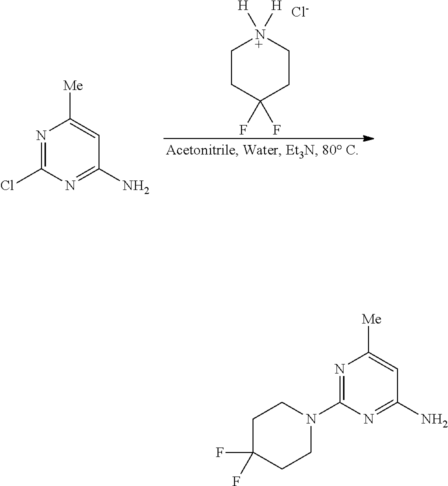 [SMILES: Cc1cc(N)nc(Cl)n1 ; FC1(F)CC[NH2+]CC1 ; [Cl-] ; Cc1cc(N)nc(N2CCC(F)(F)CC2)n1]

2-Chloro-6-methylpyrimidin-4-amine (Commercially available, 150 g, 1.0 equiv.), 4,4-difluoropiperidin-1-ium chloride (Commercially available, 189 g, 1.15 equiv.), acetonitrile (360 mL), water (240 mL) and triethylamine (148 g, 1.4 equiv.) were added to a vessel. The mixture was agitated for 10 minutes, warmed to 80° C., and agitated for 16 hours. The mixture was cooled to 5° C. and 2-(4,4-difluoropiperidin-1-yl)-6-methylpyrimidin-4-amine seed, which was obtained from previous batch, (24 gl) was added. The mixture was agitated for 15 minutes and aqueous KOH (1.1 M, 150 mL) was added. The mixture was agitated for 1 hour and aqueous KOH (1.1 M, 1.62 L) was added over 10 hours. The mixture was agitated for 1 hour and filtered. The cake was washed with a pre-agitated mixture of acetonitrile (60 mL) and water (540 mL). The cake was dried under vacuum at 60° C. and 2-(4,4-difluoropiperidin-1-yl)-6-methylpyrimidin-4-amine was isolated as crystalline solid in 89% yield. Without the crystal seed, the yield was about 1% less. 1H NMR (500 MHZ, dmso-d6): δ 6.30 (bs, 2H), 5.64 (d, 0.7 Hz, 1H), 3.79 (t, 5.8 Hz, 4H), 2.06 (s, 3H), 1.90 (tt, 14.2, 5.8 Hz, 4H); ¹³C NMR (125 MHz, dmso-d6): δ 164.4, 164.4, 160.8, 123.4 (t, ¹J_{C-F}=−240.8 Hz), 93.7, 40.1 (t, ³J_{C-F}=5.1 Hz), 33.2 (t, ²J_{C-F}=22.1 Hz), 23.7; 19F NMR (471 MHz, DMSO-d6) δ −94.7; HRMS-ESI (m/z): [M+H]⁺ calcd for [C₂₃H₂₈N₂O₅S+H]⁺, 445.1792; found: 445.1782.

### Example 7: Preparation of 2-(4,4-difluoropiperidin-1-yl)-6-methylpyrimidin-4-amine

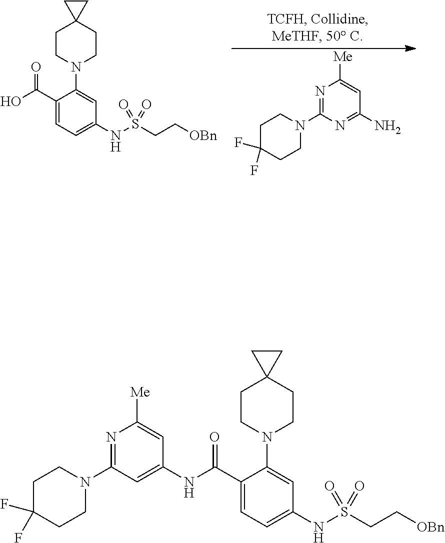 [SMILES: O=C(O)c1ccc(NS(=O)(=O)CCOCc2ccccc2)cc1N1CCC2(CC1)CC2 ; Cc1cc(N)nc(N2CCC(F)(F)CC2)n1 ; Cc1cc(NC(=O)c2ccc(NS(=O)(=O)CCOCc3ccccc3)cc2N2CCC3(CC2)CC3)cc(N2CCC(F)(F)CC2)n1]

4-((2-(benzyloxy)ethyl)sulfonamido)-2-(6-azaspiro[2.5]octan-6-yl)benzoic acid (150.3 g, 1.0 equiv), 2-(4,4-difluoropiperidin-1-yl)-6-methylpyrimidin-4-amine (90.8 g, 1.2 equiv), and chloro-N,N,N′,N′-tetramethylformamidinium hexafluorophosphate (TCFH) (139.3 g, 1.5 equiv) 2-MeTHF (1300 mL) and acetonitrile (150 mL) were added to a reaction vessel. 2,4,6-trimethylpyridine (collidine) (109 mL, 2.5 equiv) was added and the reaction mixture was agitated at 25° C. for 90 min. The reaction mixture was warmed to 50° C. and agitated for an additional 4 hours. Upon reaction completion, a 2 M NaOH solution (600 mL) was added followed by aqueous sodium chloride (600 mL, 15 wt %). The resultant mixture was agitated for 10 min at 50° C. The phases were separated, and the organic layer was further washed with an aqueous NaH₂PO₄ (1200 mL, 25 wt %). The resultant mixture was agitated for 10 min at 50° C. The phases were separated and to the organic layer was added 1200 ml of water. The resultant mixture was agitated for 10 minutes at 50° C. and the layers were separated. The organic layer was concentrated to a volume of approximately 525 mL. 2-MeTHF was added (975 mL), and the organic layer was concentrated to a volume of approximately 525 mL. 2-MeTHF was added (975 mL), and the organic layer was concentrated to a volume of approximately 525 mL. 2-MeTHF was added (975 mL) and charcoal was added (3 g, 2 wt %). The resultant suspension was agitated for 12 hours at 20° C. The suspension was filtered over CELITE® and washed with 150 mL MeTHF. The filtered solution was warmed to 50° C. The mixture was seeded with 2-(4,4-difluoropiperidin-1-yl)-6-methylpyrimidin-4-amine (1.5 g, 1 wt %), which was obtained from previous batch, and agitated for 1 h. The mixture was cooled to 35° C. and IPA (788 mL) was added over 3 hours. The mixture was agitated for an additional 1 hour and IPA (788 mL) was added over 1 h. The mixture was agitated for an additional 1 h, and cooled to 20° C. The mixture was agitated for 12 hours and filtered. The cake was washed with 2-MeTHF/IPA (25% MeTHF v/v, 600 mL) followed by IPA (600 mL, 2×). The cake was dried under vacuum at 40° C. for 24 hours. The product 2-(4,4-difluoropiperidin-1-yl)-6-methylpyrimidin-4-amine was isolated as an off-white crystalline solid (173.3 g, 80%). Without the crystal seed, the yield was about 1% less. Melting point=156-160° C. 1H NMR (500 MHz, DMSO-d6): δ 13.29 (s, 1H), 10.31 (s, 1H), 8.03 (d, J=8.6 Hz, 1H), 7.40 (s, 1H), 7.30-7.21 (m, 6H), 7.14 (dd, J=8.7, 2.1 Hz, 1H), 4.41 (s, 2H), 3.91-3.89 (m, 4H), 3.80 (t, J=5.8 Hz, 2H), 3.56 (t, J=5.9 Hz, 2H), 2.91-2.89 (m, 4H), 2.31 (s, 3H), 2.01-1.94 (m, 4H), 1.91-1.52 (m, 4H), 0.36 (s, 4H). ¹³C NMR (126 MHz, DMSO-d6): δ 169.0, 163.6, 160.2, 158.2, 153.6, 143.1, 137.7, 132.3, 128.0, 127.37, 127.35, 123.0 (t, J=241.0 Hz), 120.7, 114.4, 111.2, 98.5, 71.9, 63.7, 53.5, 51.4, 40.2, 34.2, 33.1 (t, J=22.3 Hz), 24.3, 16.8, 11.1; 19F NMR (471 MHz, DMSO-d6) δ −94.8; HRMS-ESI (m/z): [M+H]⁺ calcd for [C₃₃H₄₀F₂N₆O₄S+H]⁺, 655.2873; found, 655.2895.

### Example 8: Preparation of N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide

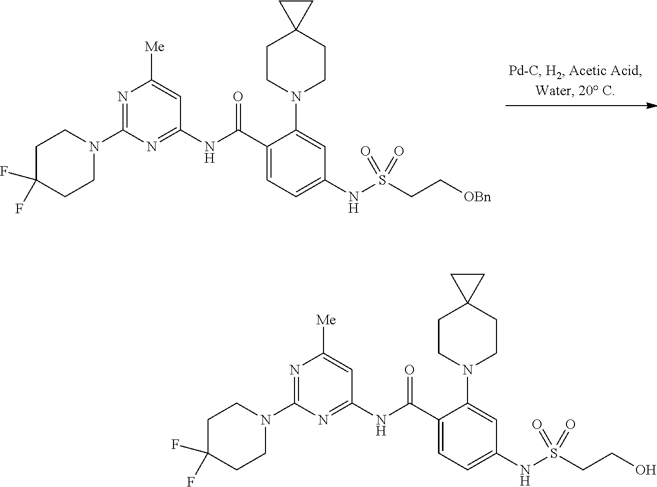 [SMILES: Cc1cc(NC(=O)c2ccc(NS(=O)(=O)CCOCc3ccccc3)cc2N2CCC3(CC2)CC3)nc(N2CCC(F)(F)CC2)n1 ; Cc1cc(NC(=O)c2ccc(NS(=O)(=O)CCO)cc2N2CCC3(CC2)CC3)nc(N2CCC(F)(F)CC2)n1]

2-(4,4-difluoropiperidin-1-yl)-6-methylpyrimidin-4-amine (700 g, 1.0 equiv.), 10% Pd/C (105 g, 15 wt %, 50% wet), acetic acid (6 L) and water (1.05 L) were added to a hydrogenation vessel. The vessel was inerted with N₂ (4 cycles) and placed under 45 psi H₂. Hydrogenation was performed at 20° C. for 25 hours. After completion of the reaction, the batch was filtered, and catalyst cake rinsed with methyl isobutyl ketone (MIBK) (4.9 L). The filtrate was washed with water (7 L) and the aqueous layer extracted twice with MIBK (2×3.5 L). The combined MIBK extracts were washed twice with aqueous NaOH (7 L, 3 wt %). The solution was concentrated to a total of 7 L and polish filtered. The polish filtration was chased with a 2 L MIBK rinse. The solution was concentrated under reduced pressure to a total of 1.5 volume, heated to 100° C., and toluene (7 L) was added. The solution was cooled to 85° C. and seeded with N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide, which was obtained from previous batch, (11 g). The mixture was agitated at 85° C. for 1 hour, cooled to 50° C. over 3 hours, and heated to 70° C. in hour. The suspension was agitated at 70° C. for 1 hour and cooled to 15° C. over 6 hours. The suspension was agitated at 15° C. for 2 hours and filtered. The cake was rinsed with 2.1 L of toluene. The solids were dried under vacuum at 40° C. N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide was isolated as a crystalline product in 85% yield. Without the crystal seed, the yield was about 1% less. 1H NMR (500 MHz, DMSO-d6) δ 13.34 (s, 1H), 10.23 (s, 1H), 8.05 (d, 8.6 Hz, 1H), 7.28 (d, 2.2 Hz, 1H), 7.14 (dd, 8.6, 2.2 Hz, 1H), 4.93 (t, 6.2 Hz, 1H), 3.90 (bt, 6.0 Hz, 4H), 3.77 (q, 6.2 Hz, 2H), 3.36 (t, 6.2 Hz, 2H), 2.97 (t, 5.3 Hz, 4H), 2.30 (s, 3H), 1.98 (tt, 13.8, 6.0 Hz, 4H), 1.72 (m, 4H), 0.38 (s, 4H) ¹³C NMR (126 MHz, DMSO-d6) δ 169.1, 163.6, 160.3, 158.3, 153.7, 143.2, 132.4, 123.1 (t, ¹J_{C-F}=−241.0 Hz), 120.7, 114.6, 111.3, 98.5, 55.4, 53.8, 53.6, 40.2 (t, ³J_{C-F}=5.0 Hz), 33.1 (t, ²J_{C-F}=22.3 Hz), 34.2, 24.4, 16.8, 11.1; 19F NMR (471 MHz, DMSO-d6) δ −94.8; HRMS-ESI (m/z): [M+H]⁺ calcd for [C₂₆H₃₄F₂N₆O₄S+H]⁺, 565.2409; found, 565.2407.

### Example 9: Preparation of N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide Hydrochloride

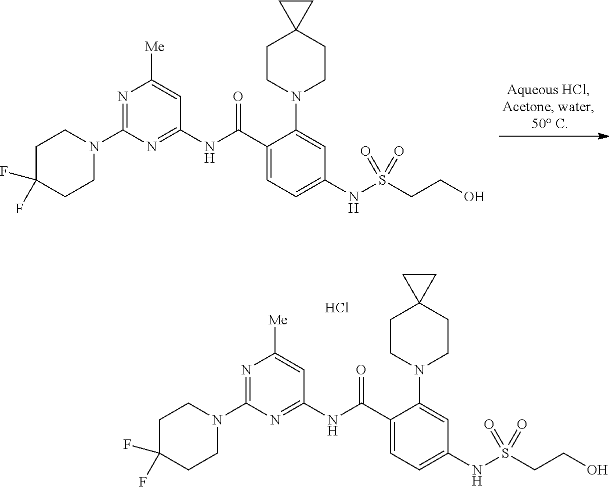 [SMILES: Cc1cc(NC(=O)c2ccc(NS(=O)(=O)CCO)cc2N2CCC3(CC2)CC3)nc(N2CCC(F)(F)CC2)n1 ; Cc1cc(NC(=O)c2ccc(NS(=O)(=O)CCO)cc2N2CCC3(CC2)CC3)nc(N2CCC(F)(F)CC2)n1 ; Cl]

N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide (100 g, 1.0 equiv.) was added 1800 mL of premixed 8/1 of acetone and water. The mixture was heated to 47° C. and agitated at that temperature for 30 minutes. The solution was polished filtered and the polish filtration chased with 450 ml of premixed 8/1 of acetone and water. To the mixture was added a solution of 150 mL of water and concentrated aqueous hydrochloric acid (1.2 equiv. of HCl, 24.3 g) over 30 minutes at 47° C. Water was added to the mixture (100 mL) and it was agitated at 47° C. for 30 minutes. The reactor was cooled to 15° C. over 4 hours. The batch was slurry-milled to reduce particle size. The product was isolated by filtration, washed with acetone (300 mL), and dried under vacuum at 40° C. to afford N-(2-(4,4-difluoropiperidin-1-yl)-6-methylpyrimidin-4-yl)-4-((2-hydroxyethyl)sulfonamido)-2-(6-azaspiro[2.5]octan-6-yl)benzamide hydrochloride as a crystalline solid in 92% yield. 1H NMR (500 MHz, DMSO-d6) δ 13.87 (bs, 1H), 10.41 (s, 1H), 8.05 (d, 8.7 Hz, 1H), 7.54 (s, 1H), 7.34 (d, 2.2 Hz, 1H), 7.19 (dd, 8.7, 2.2 Hz, 1H), 3.99 (bt, 6.1 Hz, 4H), 3.76 (t, 6.4 Hz, 2H), 3.37 (t, 6.4 Hz, 2H), 2.98 (bt, 5.3 Hz, 4H), 2.47 (s, 3H), 2.08 (btt, 13.6, 6.1 Hz, 4H), 1.69 (m, 4H), 0.39 (s, 4H); ¹³C NMR (126 MHz, DMSO-d6) δ 165.1, 164.0, 159.6, 156.6, 153.8, 143.8, 132.5, 122.6 (t, ¹J_{C-F}=−241.2 Hz), 120.1, 114.8, 111.5, 99.2, 55.4, 54.0, 53.6, 41.4, 34.2, 33.0 (t, ²J_{C-F}=22.8 Hz), 21.9, 16.8, 11.2; 19F NMR (471 MHz, DMSO-d6) δ −95.0; HRMS-ESI (m/z): [M+H]⁺ calcd for [C₂₆H₃₄F₂N₆O₄S+H]⁺, 565.2409; found, 565.2407.

The foregoing is merely illustrative of the invention and is not intended to limit the invention to the disclosed uses. Variations and changes, which are routine to one skilled in the art, are intended to be within the scope and nature of the invention, which are defined in the appended claims. All mentioned references, patents, applications and publications, are hereby incorporated by reference in their entirety, as if here written.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure.

What is claimed is:

1. A method of preparing a KIF18A inhibitor compound having the following chemical structure: 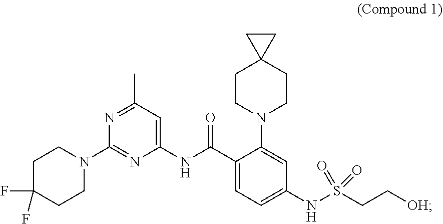 [SMILES: Cc1cc(NC(=O)c2ccc(NS(=O)(=O)CCO)cc2N2CCC3(CC2)CC3)nc(N2CCC(F)(F)CC2)n1]
comprising reacting a Compound 2 having the structure: 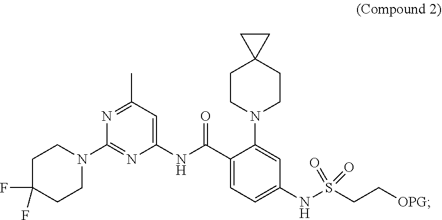
wherein PG is a hydroxyl protecting group selected from C₁₋₆alkyl, C₁₋₆alkyl-O—C₁₋₆alkyl, tetrahydropyranyl, allyl, or benzyl; with a suitable deprotection agent in a suitable solvent to form said Compound 1.

2. The method according to claim 1, further comprising preparing said Compound 2, comprising: reacting a Compound 3, having the formula: 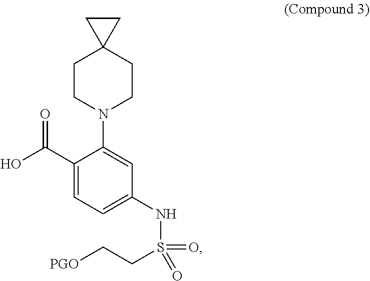
or a salt thereof; wherein PG is a hydroxyl protecting group selected from C₁₋₆alkyl, C₁₋₆alkyl-O—C₁₋₆alkyl, tetrahydropyranyl, allyl, or benzyl; with a Compound 4, having the formula 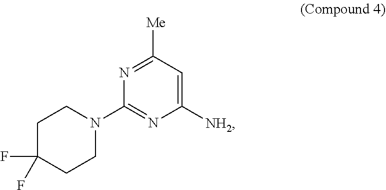 [SMILES: Cc1cc(N)nc(N2CCC(F)(F)CC2)n1]
or a salt thereof; in the presence of an amide coupling reagent and a base in an organic solvent, to form said Compound 2.

3. The method according to claim 2, further comprising preparing said Compound 3, or a salt thereof, comprising: reacting a Compound 5, having the formula: 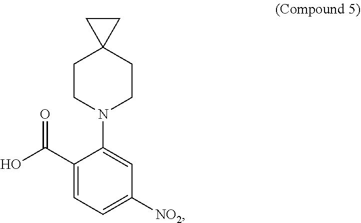 [SMILES: O=C(O)c1ccc([N+](=O)[O-])cc1N1CCC2(CC1)CC2]
or a salt thereof; with a Compound 6, having the formula 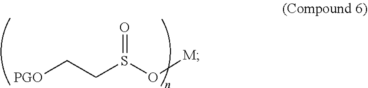
wherein PG is a hydroxyl protecting group selected from C₁₋₆alkyl, C₁₋₆alkyl-O—C₁₋₆alkyl, tetrahydropyranyl, allyl, or benzyl; M is metal, and n is an integer selected from 1 or 2; or a hydrate thereof; and a base optionally in the presence of a catalyst in an organic solvent at elevated temperature to form said Compound 3, or a salt thereof.

4. The method according to claim 3, further comprising preparing said Compound 5, or a salt thereof, comprising: reacting a Compound 7, having the formula: 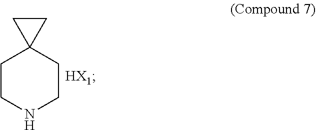
wherein X₁ is a halide; with a Compound 8, having the formula 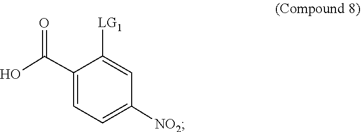
wherein LG₁ is a leaving group; in the presence of a base, in an organic solvent at elevated temperature to form said Compound 5, or a salt thereof.

5. The method according to claim 2, further comprising preparing said Compound 4, comprising: reacting a Compound 9, having the formula: 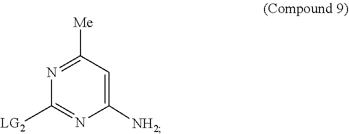
wherein LG₂ is a leaving group; with a Compound 10, having the formula 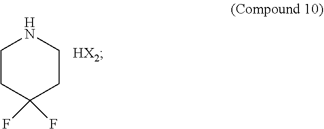
wherein X₂ is a halide; in the presence of a base at elevated temperature in an organic solvent to form said Compound 4.

6. A compound, which is 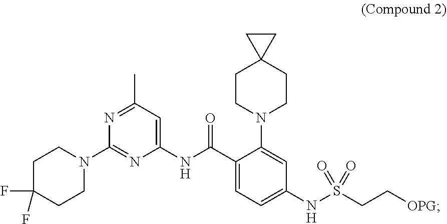
wherein PG is benzyl (Compound 2a).

7. A compound, which is 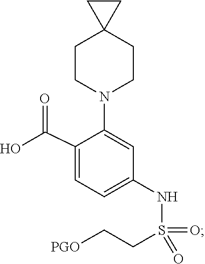
wherein PG is benzyl (Compound 3a).

8. A compound, which is 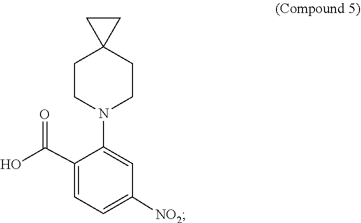 [SMILES: O=C(O)c1ccc([N+](=O)[O-])cc1N1CCC2(CC1)CC2]
or a salt thereof.

9. The method according to claim 3, further comprising preparing said Compound 6 having the following chemical structure: 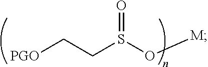
wherein PG is a hydroxyl protecting group, M is metal, and n is 1 or 2 (Compound 6); or a hydrate thereof; comprising reacting a Compound 11 having the structure 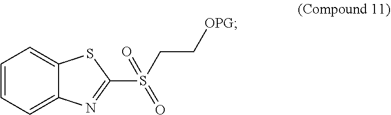
wherein PG is a hydroxyl protecting group selected from C₁₋₆alkyl, C₁₋₆alkyl-O—C₁₋₆alkyl, tetrahydropyranyl, allyl, or benzyl, with a base in a polar solvent at a moderately elevated temperature to form said Compound 6 or a hydrate thereof.

10. The method according to claim 9, further comprising preparing said Compound 11 by reacting a Compound 12 having the following chemical structure: 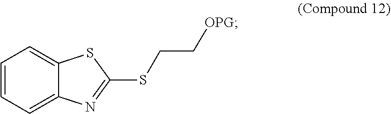
wherein PG is a hydroxyl protecting group selected from C₁₋₆alkyl, C₁₋₆alkyl-O—C₁₋₆alkyl, tetrahydropyranyl, allyl, or benzyl; with an oxidizing agent; in the presence of a catalyst, in a solvent and a slightly elevated temperature to form said Compound 11.

11. The method according to claim 10, further comprising preparing said Compound 12 by reacting a Compound 13 having the following chemical structure: 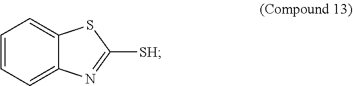
with a Compound 14 having the following chemical structure: 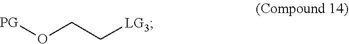
wherein PG is a hydroxyl protecting group selected from C₁₋₆alkyl, C₁₋₆alkyl-O—C₁₋₆alkyl, tetrahydropyranyl, allyl, or benzyl; and LG₃ is a leaving group; in the presence of a base, in a solvent and a moderately elevated temperature to form said Compound 12.

12. The method according to claim 1, wherein PG is a benzyl and said suitable deprotection agent is palladium on carbon catalyst; or wherein said solvent is a polar solvent.

13. The method according to claim 2, wherein:
said amide coupling reagent is selected from a chloroformamidinium salt, 2-chloro-1,3-dimethylimidazolinium chloride (DMC), 1-(chloro-1-pyrrolidinylmethylene)pyrrolidinium hexafluorophosphate (PyCIU), 2-chloro-4,6-dimethoxy-1,3,5-triazine (CDMT), or N-Ethoxycarbonyl-2-ethoxy-1,2-dihydroquinoline (EEDQ); wherein said base is selected from N-methylmorpholine (NMM), N,N-Diisopropylethylamine (DIPEA), triethylamine (TEA), 2,4,6-trimethylpyridine (collidine), or 2,6-lutidine; or
said chloroformamidinium salt is chloro-N,N,N′,N′-tetramethylformamidinium hexafluorophosphate (TCFH) and said base is 2,4,6-trimethylpyridine (collidine); or
said method is conducted at a temperature from 25° C. to 50° C., 40° C. to 50° C., or 50° C.; or
said solvent is organic solvent selected from 2-methyl THF, acetonitrile, isopropyl acetate, dichloromethane, THF, or mixture thereof; or said solvent is 2-methyl THF and acetonitrile.

14. The method according to claim 3, wherein:
said method is performed catalyst free or in the presence of a metal catalyst selected from iron, gold, or palladium catalyst; or wherein said method is performed in the presence of a metal catalyst, wherein said catalyst is iron (III) chloride hexahydrate; or
said base is sodium bisulfite; or
said elevated temperature is at solvent reflux temperature or from 60° C. to 100° C.; 70° C. to 90° C. C.; or 70° C.; or
wherein said solvent is NMP.

15. The method according to claim 4, wherein:
said base is hydroxide or amine; or wherein said base is potassium hydroxide or diisopropyl ethyl amine; or said base is potassium hydroxide; or
said solvent is polar aprotic solvent selected from NMP, DMAC, DMF, or DMSO; or said solvent is NMP; or
said elevated temperature is at solvent reflux temperature or from 80° C. to 140° C.; 100° C. to 140° C. C; or 120° C.; or
said compound 5 is crystallized by addition of an acid; or said acid is phosphoric acid.

16. The method according to claim 5, wherein said base is amine or hydroxide; or said base is triethyl amine; or
said elevated temperature is at solvent reflux temperature or from 60° C. to 100° C.; 60° C. to 85° C. C; or 80° C.; or
said solvent is acetonitrile and water mixture.

17. The method according to claim 9, wherein said base is hydroxide, or said base is calcium hydroxide, sodium hydroxide; or said base is calcium hydroxide; or
said solvent is a mixture of C₁₋₆alkyl alcohol and water; or
said Compound 6 is crystallized in acetone; or
said temperature is from 25° C. to 50° C.; from 40° C. to 50° C.; or 50° C.

18. The method according to claim 10, wherein said oxidizing agent is peroxide or peroxycarboxylic acid; or said oxidizing agent is hydrogen peroxide or meta peroxycarboxylic acid; or
said catalyst is sodium tungstate; or
said temperature is from 25° C. to 40° C.; 30° C. to 35° C. C; or 30° C.; or
said solvent is acetonitrile and water mixture; or
said Compound 12 is crystallized in acetone or mixture of acetone and water; or
said Compound 12 is not isolated and said solvent is acetonitrile.

19. The method according to claim 11, wherein said base is bicarbonate, carbonate, hydroxide, or phosphate; or wherein said base is calcium carbonate; or
said solvent is alcohol, or said solvent is methanol; or
said temperature is at solvent reflux temperature or from 70° C. to 100° C.; 75° C. to 90° C. C; or 82° C.

20. The method according to any one of claim 1-5 or 9-19, further comprising reacting said Compound 1 with an acid HA in a solvent to form a pharmaceutically acceptable salt of Compound 1 having a formula 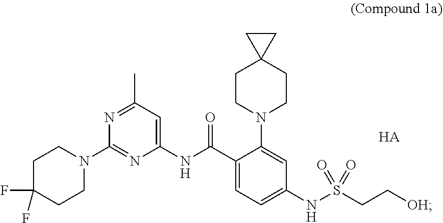 [SMILES: Cc1cc(NC(=O)c2ccc(NS(=O)(=O)CCO)cc2N2CCC3(CC2)CC3)nc(N2CCC(F)(F)CC2)n1 ; "HA"]
wherein said acid HX is HCl, methanesulfonic acid, or para toluenesulfonic acid; or said acid HX is HCl in DMSO; or wherein said solvent is water.

21. The method according to any one of claim 1-5 or 9-20, wherein said PG is benzyl.

22. A compound having a chemical structure: 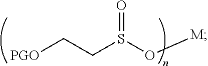
wherein PG is a hydroxyl protecting group selected from C₁₋₆alkyl, C₁₋₆alkyl-O—C₁₋₆alkyl, tetrahydropyranyl, allyl, or benzyl; preferably methyl-O-methyl, tetrahydropyranyl, or benzyl; more preferably benzyl; M is a metal; and n is an integer selected from 1, 2, or 3; or a solid form thereof.

23. The compound of claim 22, wherein PG is benzyl, M is calcium, and n is 2 (Compound 6a), having the structure 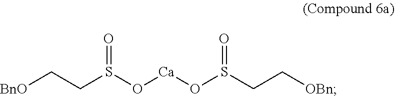
or a solid form thereof.

24. The solid form of said Compound 6a according to claim 23, which is crystalline or amorphous.

25. The solid form of said Compound 6a according to claim 24, which is a Compound 6a-I having the formula 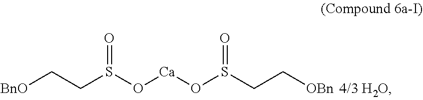
which is crystalline.

26. The crystalline Compound 6a-I according to claim 25, further characterized by XRPD pattern peaks at 4.2, 8.2, and 12.2±0.2° 2θ using Cu Kα radiation; or
further characterized by XRPD pattern peaks at 13.6, 14.2, 18.3, 19.5, 20.6, 20.9, and 22.9±0.2° 2θ using Cu Kα radiation; or further characterized by XRPD pattern peaks at 16.2, 16.7, 19.2, 21.4, 23.9, 24.4, 24.7, 25.5, 27.6, 28.1, 30.3, 33.3, and 36.6±0.2° 2θ using Cu Kα radiation; or
having an XRPD pattern substantially as shown in FIG. 1; or
having a first endothermic transition at 124.96° C. to 130.96° C.; and a second endothermic transition at 256.11° C. to 262.11° C.; as measured by Differential Scanning calorimetry; or wherein the first endothermic transition is at 127.96° C.±3° C.; and the second endothermic transition is at 259.11° C.±3° C.; or
having a Thermogravimetric Analysis (TGA) substantially as shown in FIG. 3; or
having a single crystal structure substantially as shown in FIG. 4.

27. The crystalline Compound 6a-I of any one of claims 25 to 26, which crystalline form is stable and low in hygroscopicity.
